(19) United States
(12) Patent Application Publication
(10) Pub. No.: US 2025/0208145 A1
(43) Pub. Date: Jun. 26, 2025

(54) PAPER-BASED MICROFLUIDIC CHIP FOR MEASUREMENT OF CYSTATIN C IN PLASMA AND SERUM (CYS-C PAPER CHIP)

(71) Applicant: University of Manitoba, Winnipeg (CA)

(72) Inventors: Francis Lin, Winnipeg (CA); Xiaoou Ren, Winnipeg (CA); Dumitru Tomsa, Winnipeg (CA); Amanda Stefanson, Winnipeg (CA); Yang Liu, Winnipeg (CA); Claudio Rigatto, Winnipeg (CA); Paul Komenda, Winnipeg (CA); Navdeep Tangri, Winnipeg (CA)

(21) Appl. No.: 18/848,834

(22) PCT Filed: Mar. 17, 2023
(86) PCT No.: PCT/CA2023/050356

§ 371 Date: Sep. 19, 2024

Related U.S. Application Data
(60) Provisional application No. 63/322,958, filed on Mar. 23, 2022.

(51) Int. Cl. G01N 33/68; G01N 33/543

(52) U.S. Cl. CPC G01N 33/6893; G01N 33/54346; G01N 33/54388; G01N 2333/8139; G01N 2800/347

(57) ABSTRACT

Provided are paper-based microfluidic devices for measurement of cystatin C in a biological sample, such as blood or a blood fraction, and methods for fabricating such devices. Also provided are methods of detecting cystatin C in a biological fluid sample to diagnose or monitor a chronic kidney disease.

### RELATED APPLICATION

Benefit of priority is claimed to U.S. Provisional Application No. 63/322,958, titled “PAPER-BASED MICROFLUIDIC CHIP FOR MEASUREMENT OF CYSTATIN C IN PLASMA AND SERUM (CYS-C PAPER CHIP),” filed Mar. 23, 2022.

Where permitted, the subject matter of the above-referenced application is incorporated by reference in its entirety.

### TECHNICAL FIELD

The present invention relates to paper-based microfluidic devices, and methods and systems using such devices. Provided are devices that manipulate, process, or otherwise alter micro-sized amounts of fluids and fluid samples; paper-based microfluidic analysis equipment; and to a point of care diagnostic device, for measurement of cystatin C in plasma and serum.

### BACKGROUND

In many disease states, measurement of relevant biomarkers in body fluids (urine, blood, saliva, tears) is necessary to establish diagnosis or prognosis, and to monitor progression and response to treatment. Chronic Kidney Disease (CDK) is an archetypal example of this. CKD is a global health epidemic, afflicting more than 850M people world-wide, and is a potent risk factor for kidney failure, heart disease and death. Fortunately, effective treatments exist which can prevent or delay catastrophic downstream outcomes if CKD is diagnosed early. Diagnosis of CKD requires measurement of kidney function, or more specifically, estimation of the glomerular filtration rate (GFR).

In clinical practice, GFR is usually calculated from the serum creatine concentration. Although serum creatinine is the most popular biomarker of GFR, due to widespread availability and relatively low cost, it is far from perfect, because serum creatinine level is influenced by multiple factors other than the GFR, including sex, age, race muscle mass, and nutritional status. In this regard, cystatin-C (CysC) is superior to creatinine as a GFR biomarker, because the CysC level is much less influenced by non-GFR factors. In addition, measurement of cystatin-C is of emerging interest in many non-kidney diseases including cardiovascular disease, Alzheimer's disease, diabetes and cancer. Despite its advantages, cystatin-c measurement is complex and expensive, severely limiting clinical availability and utility.

Immunologically-based laboratory methods such as traditional ELISA assays and radioimmunoassay could be used for the confirmation and measurement of CysC level in a sample of a subject's blood or serum. However, these laboratory tests are complicated to use and have high facility requirements, and the traditional ELISA well-plate method requires relatively large volumes of reagents. A point-of-care (POC) method, which optimally balances accuracy, cost, simplicity and low facility requirement, would be a highly desirable tool to effectively address the epidemic of CKD, especially in in poor, remote, underserviced regions of the world.

Accordingly, a need exists for methodologies and devices that allow for the detection and measurement of a target molecule in a biological sample of a subject that addresses the disadvantages of existing technologies.

### SUMMARY

In one aspect of the invention, provided is a paper-based microfluidic device that enables advanced sample processing, manipulation and analysis for cystatin-C in a blood or blood fraction sample that has low sample and reagent consumption, high-throughput, low-cost, portability, and that can be integrated with a reading device to provide POC detection of cystatin-C as a disease biomarker for chronic kidney disease.

Paper-based microfluidic devices can be used in a lab-on-a-chip point-of-care device. Such devices can allow medical analyses to be conducted at or near the site of the patient care.

A further object of the invention is to provide a paper-based microfluidic device that is relatively simple to manufacture and reliable to use in a test for examining if cystatin-C is present in a subject's test sample, and to provide a quantitative measurement of the amount of cystatin-C present in the sample.

According to another aspect of the invention, provided is a method for detecting cystatin-C in a biological sample to diagnose or monitor a chronic kidney disease, using a paper-based microfluidic chip provided herein.

Provided is a paper-based microfluid device that includes a paper-based substrate, and on a surface of the paper-based substrate: a conjugate pad well; a first flow channel having a first end and a distal second end and comprising a first detection point and a first control point; a second flow channel having a first end and a distal second end and comprising a second detection point and a second control point; a bridging flow channel having three connection points, a first connection point connected and in fluid communication with the first end of the first flow channel, a second connection point connected and in fluid communication with the first end of the second flow channel, and a third connection point connected and in fluid communication with the conjugate pad well, forming a fluid communication path from the conjugate pad well through each of the first flow channel and the second flow channel; a first absorbent pad well connected and in fluid communication with the distal second end of the first flow channel; and a second absorbent pad well connected and in fluid communication with the distal second end of the second flow channel. In some configurations, the first flow channel and the second flow channel can be essentially parallel. The paper-based substrate can include a cellulose ester or a nitrocellulose. In some configurations, the paper-based substrate can include a cellulose ester having a capillary flow rate of from about 75 to about 120 seconds/4 cm.

A width of the first flow channel and the second flow channel each independently can be 1 mm or less. The first flow channel and the second flow channel along the major part of their length can have the same cross-sectional shape. A length of the first flow channel and the second flow channel each independently can be in a range of about 7.0 to 15 mm. The first detection point and the second detection point can be located in a same plane and can be essentially parallel to each other. The first control point and the second control point can be located in a same plane and can be essentially parallel to each other.

The first detection point, the second detection point, the first control point, and the second control point each independently can have a shape selected from among a circle, an oval, an ellipse, a square, a rectangle, a diamond, a parallelogram, a trapezoid, a rhombus, a convex kite, a concave kite, a pentagon, a hexagon, and an octagon. In some configurations, the first detection point and the second detection point can have the same shape. In some configurations, the first control point and the second control point can have the same shape. In some configurations, the first detection point, the second detection point, the first control point, and the second control point each can have a shape that is a circle.

The first detection point and the first control point each independently can have a width that is 100% to 150% of a width of the first flow channel. The second detection point and the second control point each independently can have a width that is 100% to 150% of a width of the second flow channel. A distance between first detection point and the first control point, and a distance between the second detection point and the second control point can be 100% to 300% of the width of the flow channel in which they are located.

In some configurations, the first flow channel and the second flow channel, and the bridging flow channel, each separately can include two walls of a hydrophobic material defining each flow channel open on the surface of the paper-based substrate.

The paper-based microfluid device provided herein can further include a conjugate pad in the conjugate pad well. The conjugate pad can include or be made of glass fiber. The paper-based microfluid device provided herein can further include an absorbent pad in each of the absorbent pad wells. The absorbent pad can include or be made of cellulose fiber.

Also provided are methods of fabricating a paper-based microfluidic device. The methods can include providing a paper-based substrate having a first side and a reverse second side; printing, with a solid ink printer, a solid wax ink on the first side of the paper-based substrate to form a design of the microfluidic device on a surface of the paper-based substrate to yield a printed substrate; heating the printed substrate to a temperature above the melting point of the wax contained in the solid wax ink to melt the wax; allowing the melted wax to penetrate into at least a portion of the paper-based substrate; and cooling the printed substrate to yield the microfluidic device.

In some methods, the paper-based substrate can be a cellulose ester or a nitrocellulose. In the methods, the heating can be performed using any known method that can melt the wax in the solid wax ink. In some methods, the heating is performed by inserting the printed substrate into a heated chamber set at a temperature in the range of 60° C.-150° C.; or contacting the second side of the paper-based substrate with a heated surface set at a temperature in the range of 60° C.-150° C. The heating can be performed for a sufficient length of time to allow the wax contained in the solid wax ink to melt and to penetrate into at least a portion of the paper-based substrate. In some methods, the heating is performed for a time period of about 1 to 60 second(s).

In the methods provided herein, the design of the microfluidic device can include: an absorbent pad well; a first flow channel having a first end and a distal second end and comprising a first detection point and a first control point; a second flow channel having a first end and a distal second end and comprising a second detection point and a second control point; a bridging flow channel having three connection points, a first connection point connected to provide fluid communication with the first end of the first flow channel, a second connection point connected to provide fluid communication with the first end of the second flow channel, and a third connection point connected to provide fluid communication with the absorbent pad well; a first absorbent pad well connected to provide fluid communication with the distal second end of the first flow channel; and a second absorbent pad well connected to provide fluid communication with the distal second end of the second flow channel.

The method can further include placing a first absorbent pad into the first absorbent pad well; placing a second absorbent pad into the second absorbent pad well; and placing a conjugate pad in the conjugate pad well.

In some methods, the paper-based substrate includes or is made of a cellulose ester or a nitrocellulose. In some methods, the paper-based substrate includes or is made of a cellulose ester that has a capillary flow rate of from about 75 to about 120 seconds/4 cm.

A width of the first flow channel and the second flow channel each independently can be 1 mm or less. The first flow channel and the second flow channel can be formed so that along the major part of their length they have the same cross-sectional shape. A length of the first flow channel and the second flow channel each independently can be about 7 to 15 mm. In some methods, the length of the first flow channel and the second flow channel can be the same. In some methods, the conjugate pad includes or is made of glass fiber, and the absorbent pad includes or is made of cellulose fiber.

Also provided are methods of detecting cystatin C in a biological fluid sample to diagnose or monitor a chronic kidney disease. The methods can include providing a paper-based microfluid device as described herein; treating a conjugate pad with a treatment solution; loading a cystatin C capture antibody at the first and second detection points of the paper-based microfluid device; loading a material that binds to the anti-cystatin C capture antibody at the first and second control points of the paper-based microfluid device; blocking the first flow channel and the second flow channel of the paper-based microfluid device with a blocking solution and allowing the flow channels to dry at room temperature for a period of 1 to 24 hours; rinsing the first flow channel and the second flow channel of the paper-based microfluid device with a rinsing solution and allowing the flow channels to dry at room temperature for a period of 1 to 30 minutes; placing a treated conjugate pad in the conjugate pad well of the paper-based microfluid device; loading a detection antibody and an aliquot of a biological sample on the treated conjugate pad to produce a test fluid; allowing the test fluid to traverse the length of the first flow channel and the second flow channel; acquiring an image of the first and second detection points of the paper-based microfluid device to calculate a first signal intensity, and acquiring an image of first and second control points of the paper-based microfluid device to calculate a second signal intensity; and using the first signal intensity and the second signal intensity to calculate a concentration of the cystatin C in the biological fluid sample.

In the methods, the biological fluid can be blood or a blood fraction. The amount of the biological fluid sample loaded on the treated conjugate pad can be 10 to 30 μL. The biological fluid can be diluted with saline or a phosphate-buffered saline prior to loading on the treated conjugate pad. In the methods, the detection antibody binds cystatin C in the biological fluid sample, and the detection antibody can be an IgG antibody. The cystatin C capture antibody that is loaded at a detection point can bind to cystatin C bound to the detection antibody. The cystatin C capture antibody can be an IgG antibody. The amount of the cystatin C capture antibody loaded at the detection points of the paper-based microfluid device can be from about 0.025 to 0.1 μL of a 3 mg/mL solution of the cystatin C capture Ab. The material that binds to the detection antibody is present at the control points, and binds detection antibodies that did not react with cystatin C in the biological fluid sample, or with detection antibodies that were not captured by the cystatin C capture antibody at the detection points. The material that binds to the detection antibody can be an anti-IgG antibody. The amount of the anti-IgG antibody loaded at the first and second control points of the paper-based microfluid device can be from about 0.025 to 0.1 μL of a 2.32 mg/mL solution of the anti-IgG antibody. The detection antibody can be a gold nanoparticle-labelled anti-cystatin C antibody. The detection antibody can be an IgG antibody. The amount of the gold nanoparticle-labelled anti-cystatin C antibody loaded on the treated conjugate pad can be from about 1 to 3 μL of a 50 μg/mL solution of the gold nanoparticle-labelled cystatin C detection antibody.

In the methods, the treatment solution and the blocking solution can include bovine serum albumin. The rinsing solution can include a surfactant. Any surfactant known in the art can be used. Exemplary surfactants include polysorbates and sorbitan esters. Exemplary polysorbates include polysorbate 20 (polyoxyethylene sorbitan monolaurate, Tween® 20), polysorbate 60 (polyoxyethylene sorbitan monostearate, Tween® 60), and polysorbate 80 (polyoxyethylene sorbitan monooleate, Tween®) 80). Exemplary sorbitan esters include sorbitan monolaurate (SPAN® 20), sorbitan monostearate (SPAN®) 60), and sorbitan monooleate (SPAN® 80). The amount of surfactant in the rinsing solution can be from about 0.01 to 0.25 wt %. In some methods, the rinsing solution contains from about 0.05 to 0.15 wt % polysorbate in PBS (1×).

The methods can further include loading a washing solution onto the conjugate pad after allowing the test fluid to traverse the length of the first flow channel and the second flow channel of the microfluidic device, and allowing the washing solution to traverse the length of the first flow channel and the second flow channel prior to acquiring an image. The washing solution can include phosphate-buffered saline. The first flow channel and the second flow channel can be allowed to dry after the washing solution has traversed their length prior to acquiring an image. In the methods provided herein, the time to acquire an image from loading the detection antibody and the aliquot of a biological sample on the treated conjugate pad can be from about 15 minutes to 20 minutes.

The methods also can include subjecting the paper substrate to a treatment with a high energy plasma prior to forming the device.

Also provided is a kit that includes a paper-based microfluidic device provided herein, and instructions for the use thereof. The kit can include a detection antibody. In some kits, the detection antibody is a gold nanoparticle-labeled detection antibody.

Also provided is paper-based microfluid device that's includes a paper-based substrate, and on a surface of the paper-based substrate: a conjugate pad well; a tapering flow channel having a wide first end and a narrow distal second end, the tapering flow channel attached via its wide first end to the conjugate pad well; a detection point attached to the narrow distal second end of the tapering flow channel; a widening flow channel having a narrow first end and a wide distal second end, the narrow first end attached to the detection point; a control point attached to the wide distal second end of the widening flow channel; a discharge channel having a first end and a second end, and attached via the first end to the control point; and an absorbent pad well attached to the second end of the discharge channel.

The conjugate pad well, the tapering flow channel, the detection point, the widening flow channel, the control point, the discharge channel, and the absorbent pad well are defined by a hydrophobic material forming a hydrophobic boundary region on the paper-based substrate. The paper-based substrate can include a cellulose ester or a nitrocellulose. The paper-based substrate can be a cellulose ester substrate that has a capillary flow rate of from about 75 to about 120 seconds/4 cm. The paper-based substrate can have a surface that has been treated with a high energy plasma. The conjugate pad well can have a shape selected from the group consisting of a circle, an oval, an ellipse, a square, a rectangle, a diamond, a parallelogram, a trapezoid, a rhombus, a convex kite, a concave kite, a pentagon, a hexagon, and an octagon, and can have a diameter or largest cross-sectional length that is in the range of 4-11 mm. The absorbent pad well can have a shape selected from the group consisting of a circle, an oval, an ellipse, a square, a rectangle, a diamond, a parallelogram, a trapezoid, a rhombus, a convex kite, a concave kite, a pentagon, a hexagon, and an octagon, and can have a diameter or largest cross-sectional length that is in the range of 4-11 mm.

The microfluid device provided herein can have a tapering flow channel that has a length of about 4 to 10 mm; a width at the wide first end from about 0.75 mm to 1.5 mm; and a width at the narrow distal second end of about 0.25 mm to 0.5 mm. A ratio of the width at the wide first end of the tapering flow channel to the width at the narrow distal second end of tapering flow channel can be from 2.75:1 to 3.25:1. The detection point can have a shape selected from the group consisting of a circle, an oval, an ellipse, a square, a rectangle, a diamond, a parallelogram, a trapezoid, a rhombus, a convex kite, a concave kite, a pentagon, a hexagon, and an octagon, and can have a diameter or largest cross-sectional length that is about 1.5 times to about 4.4 times the width of the widest flow path to which it is connected. The control point can have a shape selected from the group consisting of a circle, an oval, an ellipse, a square, a rectangle, a diamond, a parallelogram, a trapezoid, a rhombus, a convex kite, a concave kite, a pentagon, a hexagon, and an octagon, and can have a diameter or largest cross-sectional length that is about 1.5 times to about 4.4 times the width of the widest flow path to which it is connected. In some configurations, the detection point has a diameter or largest cross-sectional length that is in a range of 1 mm to 5 mm; and the control point has a diameter or largest cross-sectional length that is in a range of 1 mm to 5 mm.

The widening flow channel can have a length of about 4 to 10 mm; a width at the narrow first end that is from about 0.25 mm to 0.5 mm; and a width at the wide distal second end that is from about 0.75 mm to 1.5 mm. A ratio of the width at the narrow first end of the widening flow channel to the width at the wide distal second end of widening flow channel can be from 1:2.75 to 1:3.25. The discharge channel can have a length in a range of about 4.0 mm to 10 mm, and a width in a range of from about 0.75 mm to 1.5 mm.

One, or two or all three of the tapering flow channel, the widening flow channel, and the discharge channel can be treated with a blocking agent. The blocking agent can include bovine serum albumin. A conjugate pad can be included in the conjugate pad well. The conjugate pad can include glass fiber. An absorbent pad can be included in the absorbent pad well. The absorbent pad can include cellulose fiber.

Also provided is a method of fabricating a paper-based microfluidic device, the method including: providing a paper-based substrate having a first side and a reverse second side; treating the first side of the paper-based substrate with a high energy plasma generated using radio frequency (RF) electromagnetic radiation at 8-12 MHz for a time period of about 3 to 6 minutes to produce a substrate having a plasma-treated first side; printing, with a solid ink printer, a solid wax ink on the plasma-treated first side of the paper-based substrate to form a design of the microfluidic device on the plasma-treated first side of the paper-based substrate to yield a printed substrate; heating the printed substrate to a temperature above the melting point of the wax contained in the solid wax ink to melt the wax; allowing the melted wax to penetrate into at least a portion of the paper-based substrate; and cooling the printed substrate to yield the microfluidic device. The heating can be performed by: inserting the printed substrate into a heated chamber set at a temperature in the range of 60° C.-150° C.; or contacting the second side of the paper-based substrate with a heated surface set at a temperature in the range of 60° C.-150° C. The heating can be performed for a time period of about 1 to 60 second(s).

In the methods provided herein, the design of the microfluidic device can include a conjugate pad well; a tapering flow channel having a wide first end and a narrow distal second end, the tapering flow channel attached via its wide first end to the conjugate pad well; a detection point attached to the narrow distal second end of tapering flow channel; a widening flow channel having a narrow first end and a wide distal second end, the narrow first end attached to the detection point; a control point attached to the wide distal second end of the widening flow channel; a discharge channel having a first end and a second end, and attached via the first end to the control point; and an absorbent pad well attached to the second end of the discharge channel.

The methods can further include placing an absorbent pad into absorbent pad well; and placing a conjugate pad in conjugate pad well. The conjugate pad 2650 can include glass fiber, and the absorbent pad can include cellulose fiber. The conjugate pad well can have a shape selected from the group consisting of a circle, an oval, an ellipse, a square, a rectangle, a diamond, a parallelogram, a trapezoid, a rhombus, a convex kite, a concave kite, a pentagon, a hexagon, and an octagon, and a diameter or largest cross-sectional length that is in the range of 4-11 mm. The absorbent pad well can have a shape selected from the group consisting of a circle, an oval, an ellipse, a square, a rectangle, a diamond, a parallelogram, a trapezoid, a rhombus, a convex kite, a concave kite, a pentagon, a hexagon, and an octagon, and a diameter or largest cross-sectional length that is in the range of 4-11 mm.

The paper-based substrate can be or include a cellulose ester or a nitrocellulose. The cellulose ester substrate can have a capillary flow rate of from about 75 to about 120 seconds/4 cm.

In the methods, the tapering flow channel can have a length of about 4 to 10 mm; a width at the wide first end that is from about 0.75 mm to 1.5 mm; and a width at the narrow distal second end that is from about 0.25 mm to 0.5 mm. A ratio of the width at the wide first end of the tapering flow channel to the width at the narrow distal second end of tapering flow channel can be from 2.75:1 to 3.25:1. The detection point can have a shape selected from the group consisting of a circle, an oval, an ellipse, a square, a rectangle, a diamond, a parallelogram, a trapezoid, a rhombus, a convex kite, a concave kite, a pentagon, a hexagon, and an octagon, and a diameter or largest cross-sectional length can be about 1.5 times to about 4.4 times the width of the widest flow path to which it is connected, The control point can have a shape selected from the group consisting of a circle, an oval, an ellipse, a square, a rectangle, a diamond, a parallelogram, a trapezoid, a rhombus, a convex kite, a concave kite, a pentagon, a hexagon, and an octagon, and a diameter or largest cross-sectional length can be about 1.5 times to about 4.4 times the width of the widest flow path to which it is connected. The detection point can have a diameter or largest cross-sectional length that is in a range of 1 mm to 5 mm, and the control point can have a diameter or largest cross-sectional length that is in a range of 1 mm to 5 mm.

In the methods provided, the widening flow channel can have a length of about 4 to 10 mm; a width at the narrow first end that is from about 0.25 mm to 0.5 mm; and a width at the wide distal second end that is from about 0.75 mm to 1.5 mm. A ratio of the width at the narrow first end of the widening flow channel to the width at the wide distal second end of widening flow channel can be from 1:2.75 to 1:3.25. The discharge channel can have a length in a range of about 4.0 mm to 10 mm, and a width in a range of from about 0.75 mm to 1.5 mm. The methods provided herein can further include treating one, or a combination or all of the tapering flow channel, the widening flow channel, and the discharge channel with a blocking agent. The blocking agent can include bovine serum albumin.

Also provided is a method of detecting cystatin C in a biological fluid sample to diagnose or monitor a chronic kidney disease, the method including providing a paper-based microfluid device having a design shown in FIG. 7A; treating the conjugate pad with a treatment solution; loading a capture antibody at the detection point 2400, wherein the capture antibody is an anti-cystatin C antibody; loading a material that binds to a detection antibody at the control point; blocking the tapering flow channel and the widening flow channel with a blocking solution and allowing the channels to dry at room temperature for a period of 1 to 24 hour(s); rinsing the tapering flow channel and the widening flow channel with a rinsing solution and allowing the channels to dry at room temperature for a period of 1 to 30 minute(s); placing the treated conjugate pad in the conjugate pad well; loading the detection antibody and an aliquot of a biological fluid sample on the treated conjugate pad to produce a test fluid; allowing the test fluid to traverse the length of the tapering flow channel, the widening flow channel, and the discharge channel; acquiring an image of the detection point to calculate a first signal intensity, and acquiring an image of the control point to calculate a second signal intensity; and using the first signal intensity and the second signal intensity to calculate a concentration of the cystatin C in the biological fluid sample. In the methods, the biological fluid sample can be blood or a blood fraction. An amount of the biological fluid sample loaded on the treated conjugate pad can be 10 μL to 30 μL.

In the methods provided, the capture antibody can be an IgG antibody. An amount of the capture antibody loaded at the detection point can be from about 0.025 to 0.1 μL of a 3 mg/mL solution of the capture Ab. The material that binds to the detection antibody loaded at control point can be an anti-IgG antibody. An amount of the anti-IgG antibody loaded at the control point can be from about 0.025 to 0.1 μL of a 2.32 mg/mL solution of the anti-IgG antibody. The detection antibody can be a gold nanoparticle-labelled anti-cystatin C antibody. The detection antibody can be an IgG antibody. An amount of the gold nanoparticle-labelled anti-cystatin C antibody loaded on the treated conjugate pad with the biological fluid sample can be from about 1 to 3 μL of a 50 μg/mL solution of the gold nanoparticle-labelled cystatin C detection antibody. Each of the treatment solution and the blocking solution can include bovine serum albumin. The rinsing solution can include a surfactant.

The methods can further include loading a washing solution onto the conjugate pad after allowing the test fluid to traverse the length of the tapering flow channel, the widening flow channel, and the discharge channel 2550 prior to acquiring an image. The methods also can include allowing the tapering flow channel, the widening flow channel, and the discharge channel to dry after the washing solution has traversed their length prior to acquiring an image. A time to acquire an image from loading the detection antibody and the aliquot of a biological fluid sample on the treated conjugate pad can be from about 15 minutes to 20 minutes.

Also provided is kit that includes a microfluidic device as described herein having a design shown in FIG. 7A, and instructions for the use thereof. The kit can include a detection antibody. The detection antibody can a gold nanoparticle-labelled anti-cystatin C antibody.

### BRIEF DESCRIPTION OF DRAWINGS

The accompanying figures are included to provide a further understanding of the present invention, and are incorporated in and constitute a part of this specification. The drawings illustrate exemplary embodiments of the present invention and, together with the description, serve to explain principles of the present invention.

FIG. 1A is a schematic plan view of one exemplary embodiment of the microfluidic device of the present invention. FIG. 1B is a schematic plan view of one exemplary embodiment of the microfluidic device of the present invention assembled for use, with a conjugate pad in the conjugate pad well, and an absorbent pad in each of the absorbent pad wells.

FIG. 2A is a schematic plan view of a comparative paper-based microfluidic device, and FIG. 2B is a plan view of an exemplary embodiment of the microfluidic device of the present invention.

FIG. 3 is a photograph of a modified microfluid device having the design shown in FIG. 1A adhered to a substrate having an adhesive surface.

FIG. 4A is a photograph of a plan view showing the color signal observed for a comparative paper-based microfluidic device testing for cystatin C in a serum sample.

FIG. 4B is a photograph of a plan view showing the color signal observed for the paper-based microfluidic device provided herein having the design shown in FIG. 1A testing for cystatin C in a serum sample.

FIG. 5 is a graph showing a calibration curve for the cystatin-C detection for the paper-based microfluidic device provided herein having the design shown in FIG. 1A.

FIG. 6 is a photograph showing results using the paper-based microfluid chip testing blood samples from patients who have been diagnosed with CKD.

FIG. 7A is a schematic plan view of another exemplary embodiment of the microfluidic device of the present invention. FIG. 7B is a schematic plan view of another exemplary embodiment of the microfluidic device of the present invention assembled for use, with a conjugate pad in the conjugate pad well, and an absorbent pad in the absorbent pad well.

FIG. 8 is a photograph of a plan view showing the color signal observed for the paper-based microfluidic device having the design shown in FIG. 7A testing for cystatin C in a serum sample.

FIG. 9 is a graph showing a calibration curve for the cystatin-C detection for the paper-based microfluidic device provided herein having the design shown in FIG. 7A.

### DETAILED DESCRIPTION

### Definitions

Unless defined otherwise, all technical and scientific terms used herein have the same meaning as is commonly understood by one of skill in the art to which the inventions belong. All patents, patent applications, published applications and publications, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

As used herein, the singular forms “a,” “an” and “the” include plural referents unless the context clearly dictates otherwise.

As used herein, all ranges include the upper and lower limits. As used herein, the recitation of a numerical range for a variable is intended to convey that the variable can be equal to any value(s) within that range, as well as any and all sub-ranges encompassed by the broader range. Thus, the variable can be equal to any integer value or values within the numerical range, including the endpoints of the range. As an example, a variable which is described as having values between 0 and 10, can be 0, 4, 2-6, 2.75, 3.3-4.4, etc.

As used herein, “about” is a term of approximation and is intended to include minor variations in the literally stated amounts, as would be understood by those skilled in the art. Such variations include, for example, standard deviations associated with techniques commonly used to measure the amounts of the constituent elements or components of a composition or composite material, or other properties and characteristics. All of the values characterized by the above-described modifier “about,” are also intended to include the exact numerical values associated therewith. Hence “about 5 percent” means “about 5 percent” and also “5 percent.”

As used herein, “optional” or “optionally” means that the subsequently described event or circumstance does or does not occur, and that the description includes instances where said event or circumstance occurs and instances where it does not. For example, an optional component in a system means that the component may be present or may not be present in the system.

As used herein, the terms “comprises” and “comprising” are inclusive and open ended, and not exclusive. When used in the specification and claims, the terms “comprises” and “comprising” and variations thereof mean the specified features, steps or components are included, but do not exclude other features, steps or components.

Any compositions described herein are intended to encompass compositions which consist of, consist essentially of, as well as comprise, the various constituents identified herein, unless explicitly indicated to the contrary.

In the specification and claims, the singular forms include plural referents unless the context clearly dictates otherwise. As used herein, unless specifically indicated otherwise, the word “or” is used in the “inclusive” sense of “and/or” and not the “exclusive” sense of “either/or.”

As used herein, the term “exemplary” means “serving as an example or illustration,” and should not be construed as being preferred or advantageous over other configurations disclosed herein.

Unless indicated otherwise, each of the individual features or embodiments of the present specification are combinable with any other individual feature or embodiment that are described herein, without limitation. Such combinations are specifically contemplated as being within the scope of the present invention, regardless of whether they are explicitly described as a combination herein.

As used herein, the term “subject” includes members of the animal kingdom including but not limited to human beings.

As used herein, the fluid introduced into a microfluidic device for testing can be referred to a “sample fluid.”

As used herein, “analyte” means a substance of interest whose presence is being identified or whose concentration is being measured.

As used herein, “essentially parallel” means exactly parallel as well as an angle of variance between the two points is less than 15°, preferably less than 10° and more preferably less than 5°.

As used herein, a “microfluidic device” refers to a system, device, or device component that contains a liquid pathway configured to hold a fluid and to have at least one physical dimension, such as a width, of 1500 microns or less. In some configurations, a liquid pathway configured to hold a fluid has at least one physical dimension, such as a width, of 1000 microns or less.

As used herein, “paper chip” refers to a paper-based microfluidic device.

As used herein, “room temperature” refers to a temperature of about 20° C.

As used herein, “high energy plasma” refers to plasma containing ionized particles produced when a gas is subjected to a high frequency oscillating magnetic field under reduced pressure, which ionizes the gas molecules resulting in formation of a high energy plasma.

### Paper-Based Microfluidic Device

Microfluidics has been applied to a wide range of applications, but device fabrication remains a significant challenge due to economics and scalability. Some microfluid devices use techniques similar to those used to form printable circuit boards, or lithographic methods. For example, a stamp or a wafer die containing a desired pattern of a microfluid device, can be used to produce the desired features of a microfluid device. These techniques can require special expertise and equipment for manufacturing such devices.

Paper-based microfluidic devices, on the other hand, use microfluidic channels formed on or within a paper substrate, and can serve as straightforward and low-cost approach for microfluid manipulation for use in diagnostic testing devices. A major advantage compared to other techniques of fabricating microfluidic devices is that existing low-cost materials and devices can be used to fabricate the paper-based microfluidic device provided herein. A solid wax ink printer can be used to form the channels of the microfluidic device, using known materials and methods of printing solid wax ink. The substrates to be used in the device are well characterized and commercially available.

The paper-based microfluidic devices can be inexpensively and efficiently manufactured on paper-based substrates using known, reliable, and scalable printing methods such as solid wax ink printing. The fabrication techniques described herein can be scaled to larger scale processes.

Previous attempts at optimizing paper-based microfluidic devices have faced obstacles related to limitations on detection of colorimetric results because of the small volumes of analyte used, and perceived difficulty in performing multi-step assays such as sandwich ELISA assays.

The paper-based microfluidic devices provided herein overcome these challenges. It has been determined that appropriate selection of device design, fluid, reaction, and signal control, allows for low reagent use, and enhanced colorimetric signal reading is achievable. The paper-based microfluidic devices provided herein can achieve the desired detection range and sensitivity suitable for point-of-care diagnostic applications.

It has been determined that regulation of the channel width and length, and control of the flow properties of the paper substrate, can provide control of the time of arrival of analyte at specific points on the device, so that sandwich ELISA assays can be performed. Based on the hydrodynamics of the analyte and the flow properties of the paper substrate, microfluidic flow velocity can be adjusted by selecting different channel widths and depths, or modifying fluid or wall surface tension, or any combination thereof, to increase or decrease capillary force driving fluid flow through the device.

The present invention provides paper-based microfluidic devices that can simply and accurately control a fluid flow through the device. Flow through the device is due to capillary forces. The capillarity for a given liquid through a channel is the capillary forces which pull the liquid along the channel. The capillarity is dependent on the geometrical dimensions of the channel, the surface tension of the channel, the surface tension of the test fluid, and the flow properties of the paper substrate.

### First Exemplary Device

The instant Applicants determined that, in one embodiment, a compact paper-based microfluidic device can be provided by including a single conjugate pad well, two absorbent pad wells, and two flow channels connecting the conjugate pad well to the two absorbent pad wells so that the conjugate pad is in fluid communication with the two absorbent pad wells. The flow channels can have the same configuration, or a first flow channel can have a configuration that is different than a second flow channel. In some configurations, the two flow channels are identical. Each flow channel can include one or more detection points and one or more control points. Having each flow path attached to a separate absorbent pad can increase the solution absorbent capacity of the device.

The flow channels can have a width that is about 1.5 mm or less (1500 μm or less). In some embodiments, the flow channels can have a width that is about 1 mm or less (1000 μm or less). If the flow channels have a width that is greater than about 1.5 mm, such as 2 mm, the flow rate in the channels can be too fast, decreasing the binding efficiency of different reagents at the one or more detection points and one or more control points. The slower flow rate can result in higher binding efficiency. The higher binding efficiency can result in improved signal detection. In some configurations, the higher binding efficiency can result in improved color signal and colorimetric detection.

It also was determined that efficiency of the paper-based microfluidic device can be increased by decreasing the length of the flow channels to be 15 mm or less. The length of the two flow channels can be the same, or the length of the two channels can be different. The length of each of the two flow channels independently can be in the range of about 7 to 15 mm. The length of each of the two flow channels independently can be in the range of about 7.0 to 15 mm, 7.1 to 14 mm, or 7.15 to 13.5 mm, or 7.2 to 13 mm, or 7.25 mm to 12 mm, or 7.3 to 11 mm, or 7.35 to 10.5 mm, or 7.4 to 10 mm, or 7.45 to 9.5 mm. In some configurations, the length of each flow channel independently can be about 7.0, 7.25 mm, 7.5 mm, 7.75 mm, 8 mm, 8.25 mm, 8.5 mm, 8.75 mm, 9 mm, 9.25 mm, 9.5 mm, 9.75 mm, 10 mm, 10.5 mm, 11 mm, 11.5 mm, 12 mm, 12.5 mm, 13 mm, 13.5 mm, 14 mm, 14.5 mm, or 15 mm. In some configurations, the length of each flow channel is the same, and is in the range of 7.0 to 15 mm, or 7.25 to 10 mm, or 7.25 to 9.25 mm, 7.5 to 9.5 mm, or 8 to 9.25 mm, or 9 to 10 mm.

Each channel has dimensions so that liquid such as blood, serum, urine, and/or water at room temperature (at about 20° C.) can flow from the conjugate pad well to the absorbent pad by capillary forces only. In some configurations, each flow channel along the major part of their length have essentially the same cross-sectional shape and size. The first and second flow channels can be parallel or significantly parallel to each other.

The test points (detection point(s) and each control point(s)) can have any shape. The test points can have a shape that is a circle, an oval, an ellipse, a square, a rectangle, a diamond, a parallelogram, a trapezoid, a rhombus, a convex kite, a concave kite, a pentagon, a hexagon, or an octagon. The shape of each test point can be selected independently. For example, one test point can be in the shape of a circle, and one test point can be in the shape of a ellipse. In some configurations, at least one test point has a circular shape. In some configurations, all test points have the same shape. In some configurations, all test points are in the shape of a circle.

The width of the longest bisection of a test point can be independently selected. In some configurations, each detection point and each control point independently can have a width that is 100% to 150% of the width of the flow path in which each is located. For example, if a width of a flow path is 1 mm, the width of a detection point and a control point in that flow path can be in the range of 1.1 to 1.5 mm. Each detection point and each control point independently can have a longest length that is 100% to 150% of the width of the flow path in which each is located. For example, if a width of a flow path is 1 mm, the longest length of a detection point and a control point in that flow path can be in the range of 1 to 1.5 mm. For a test point that has a shape of a circle, the diameter of the circle can be in the range of 100% to 150% of the width of the flow path in which it is located. By controlling the size of each detection point and each control point, the amount of reagent required can be controlled. When the size of each detection point and each control point is within the recited ranges, the device requires less reagent consumption.

The distance between a detection point and a control point in a flow path can be selected to allow separation of the detection signals from the detection point and the control point. The distance between a detection point and a control point can be in a range of from about 150% to 300% of the width of the flow channel. For example, if a width of a flow path is 1 mm, the distance between a detection point and a control point in that flow path can be in the range of 1 to 3 mm. In some configurations, the distance between a detection point and a control point in a flow path is 1 to 3 mm, or 1.25 to 2.75 mm, or 1.5 to 2.5 mm, or 1.75 to 2.25 mm, or 1 to 2 mm, or 2 to 3 mm.

Each detection point and each control point of the paper-based microfluidic device can include a reagent that is capable of binding to an analyte in a test liquid. Examples of reagents include antibodies, antigens, enzymes, nanoparticles, and conjugates or combinations thereof. In some configurations, a detection point or a control point or both can include two or more reagents. The reagent can, e.g., be applied at detection point or a control point and be allowed to dry, such as it is well known in the art to prepare a microfluidic device with reagents.

The test liquid can be a biological fluid. The test liquid can be a mixture of a biological fluid and additional components. Exemplary biological fluids include blood and plasma. The biological fluid can be diluted with a solvent, such as water, saline, or phosphate buffered saline

In one embodiment, provided is a low-cost paper chip for precise measurement of cystatin C concentration in a drop of blood. Specifically, provided is a paper-based microfluidic device based on a sandwich-lateral flow assay, to measure cystatin C concentration in a blood sample. Loading volume, flow speed and rate are optimized by the unique design of the paper chip (e.g., paper material and channel). Test points in the paper chip allow optical colorimetric (e.g., converted color intensity) of an antigen-antibody reaction at a defined time point to determine the amount of the cystatin C concentration in the sample.

In comparison to existing enzyme-linked immunosorbent assay (ELISA) kits for measurement of cystatin C in a sample, which require highly trained lab personnel and dedicated equipment such as a plate reader, the paper-based microfluidic device provided herein has the following notable advantages: 1) low cost, 2) easy operation, 3) quantitative and stable measurement, and 4) compatibility for portable and inexpensive optical readers.

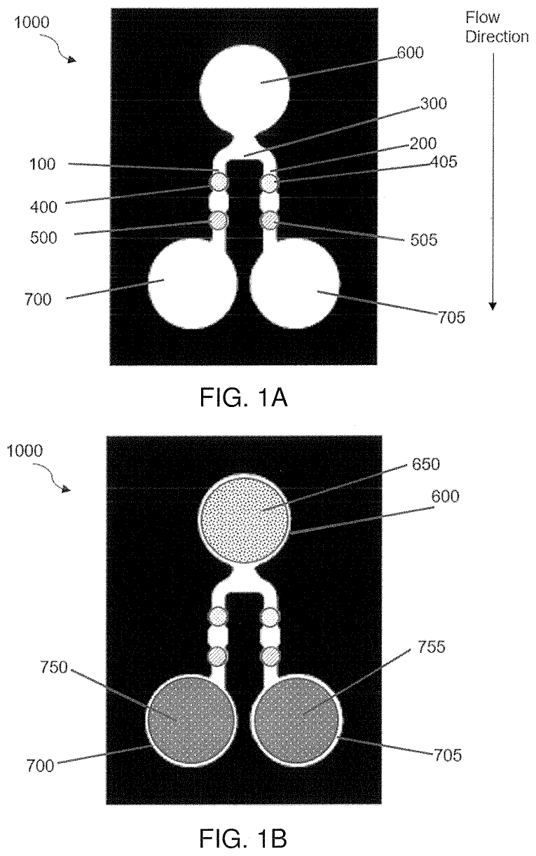

Provided is a low-cost paper-based microfluid device (paper chip) for precise measurement of cystatin C concentration in a drop of blood. A first exemplary device is depicted in FIGS. 1A and 1B.

A first exemplary embodiment of the paper-based microfluidic device provided herein can include two flow channels each including a first end and a distal second end. The flow channels can be defined by printed wax on a surface of the substrate, that, when the wax is melted, forms a barrier that prevents fluid flow outside of each flow channel.

The first end of the first flow channel and the first end of the second flow channel are connected to each other via a bridging flow channel. The bridging flow channel also is connected to a conjugate pad, forming a fluid communication path from the conjugate pad through each of the first flow channel and the second flow channel. The distal second end of the first flow channel is connected to a first absorbent pad, so that the first absorbent pad is in fluid communication with the conjugate pad via the first parallel flow channel. The distal second end of the second flow channel is connected to a second absorbent pad, so that the second absorbent pad is in fluid communication with the conjugate pad via the second parallel flow channel. Placement of analyte on the conjugate pad results in capillary flow of the analyte through the bridging flow channel and into each of the first flow channel and the second flow channel, flowing to each of the absorbent pads.

The absorbent pads can serve as waste reservoirs, as well as a driving force for capillary flow.

The first flow channel and the second flow channel each can include at least one detection point. The first flow channel and the second flow channel each can include at least one control point. In some configurations, the first flow channel and the second flow channel each include a detection point (T1 point) and control point (T2 point). The detection point in one flow channel can be essentially parallel to the detection point in the other flow channel, so that they are aligned in the same plane. In this configuration, a single detection device can view and analyze both detection points simultaneously. The control point in one flow channel can be essentially parallel to the control point in the other flow channel, so that they are aligned in the same plane. In this configuration, a single detection device can view and analyze both control points simultaneously.

The detection point of the microfluidic device includes a reaction part that includes a material reacting with an analyte or predetermined target molecule or material contained in the biological sample to generate a detectable signal.

The detectable signal can be, e.g., a fluorescent signal, a colorimetric signal, an electrochemical signal, or a combination thereof.

In one embodiment, the detectable signal is generated by gold nanoparticles conjugated to an antibody to form a labeled detection antibody.

The labelled detection antibodies on, or added to, the conjugate pad can capture the corresponding biomarker (e.g., Cystatin C) from the biological sample to form a complex, and the complex flows through the flow channel to the absorbent pad. Along the flow channel, the fluid containing the complex first encounters a capture antibody (an antibody that binds to the detection antibody, typically binding to the biomarker, such as cystatin C) at a detection point (T1) capturing the complex, while any uncaptured complex or uncomplexed detection antibody passes through and along the flow channel to a control point (T2), where it binds to an anti-IgG antibody that binds to the IgG of the detection Ab. The detectable signal color comes from gold nanoparticles conjugated onto the detection antibody, and their accumulation at T1 and T2, respectively, showing the final color intensity, which is the measurable detectable signal. The color signal at the detection point (T1) is based on the Cystatin C concentration in the biological sample, with higher concentrations producing stronger color intensity. The color signal at the control point (T2) is based on the binding of IgG (from the detection Ab) and an anti-IgG antibody, thus even with no Cystatin C in the biological sample, a color signal would be displayed at the control point (T2) due to the detection antibody. This is a reason why T2 is referred to as a control point because the control signal generated at that location can be used to confirm the assay itself is working properly.

In use, after about 1 minute after loading the sample onto the microfluidic device provided herein, a color signal is visually detectable. After about 4-5 minutes of loading the sample onto the microfluidic device, the color signal at the detection point (T1) and control point (T2) are well defined and by visual inspection appear stable. Washing the channels with buffer removes any unbound floating gold nanoparticles in the channels and at the detection point and the control point that could contribute to the color signal. The channels then are allowed to dry before measuring the color signal. Thus, a stable, consistent, quantitative measurement of the color signal can be taken in about 15 to 20 minutes after loading the sample onto the microfluidic device provided herein.

A detection device can detect, read, analyze, quantitate or any combination of these the detectable signal. For example, the detectable signal can be read by a microscope. The microscope can be a digital microscope. The microscope can be a handheld or portable microscope. The microscope can be a USB microscope. The microscope can be a fluorescent microscope. In some configurations, a portable imaging system can be used. A portable imaging system could simplify detection and can be used in a POC test device. A portable imaging system could include photodiode detector. Advances in camera technologies, such as those now incorporated into many smartphones, also could be configured for use in a portable imaging system for detection of the detectable signal. Any optical readers known in the art can be adapted for use to detect the detectable signal.

### Second Exemplary Device

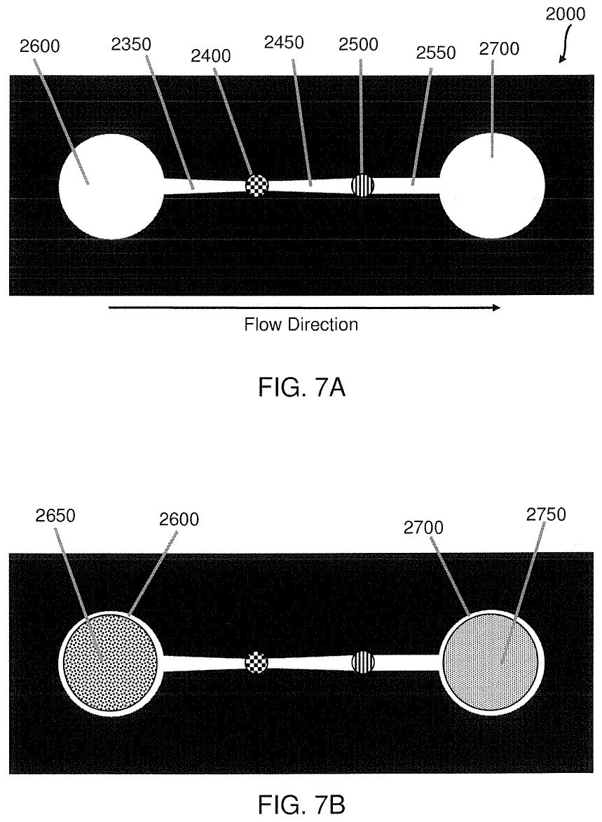

Also provided is a second low-cost paper-based microfluid device (paper chip) for precise measurement of cystatin C concentration in a drop of blood. The second exemplary device is depicted in FIGS. 7A and 7B.

The second exemplary embodiment of the paper-based microfluidic device provided herein can include a combination of a tapering flow channel and a widening flow channel. The tapering flow channel connects the conjugate pad in the conjugate pad well with a detection point, and the widening flow channel connects the detection pint to a control point. The design also includes a discharge channel that connects the control point with the absorbent pad in the absorbent pad well. The tapering flow channel, the widening flow channel, and the discharge channel provide a fluid pathway so that the conjugate pad in the conjugate pad well is in fluid communication with the absorbent pad in the absorbent pad well. The flow channels can be defined by printed wax on a surface of the substrate, that, when the wax is melted, forms a barrier that prevents fluid flow outside of each flow channel.

The first end (wide end) of the tapering flow channel is connected to a conjugate pad in a conjugate pad well, forming a fluid communication path from the conjugate pad through the tapering channel. The distal second end of the tapering flow channel (the narrow end) is connected to a detection point, so that the detection point is in fluid communication with the conjugate pad in the conjugate pad well. Placement of analyte on the conjugate pad results in capillary flow of the analyte through the tapering flow channel to the detection point. The tapered design of the tapering flow channel controls the fluid flow. Due to the decreasing width of the tapering flow channel as it approaches the detection point, flow of fluid containing antigen slows down and thus antigen capture by the antibody at the detection point can improve. Fluid flow then speeds up once passed the detection point due to the increasing width of the widening flow channel that connects the detection point to the control point. The instant Applicants determined that a compact paper-based microfluidic device can be provided by including the combination of a tapering flow channel and a widening flow channel.

The flow channels can have a width that is about 0.75 to 1.5 mm at its widest point. If the flow channels have a width that is greater than about 1.5 mm, such as 2 mm, the flow rate in the channels can be too fast, decreasing the binding efficiency of different reagents at the one or more detection points and one or more control points. The slower flow rate can result in higher binding efficiency. The higher binding efficiency can result in improved signal detection. The higher binding efficiency can result in improved signal production and improved signal detection. In some configurations, the higher binding efficiency can result in improved color signal production and improved colorimetric detection.

In the tapering flow channel, the width at the widest end of the tapering flow channel, which is the end connected to the conjugate pad well, can be from about 0.75 mm to 1.5 mm. The width of the widest end of the tapering flow channel can be about 0.75 mm, 0.8 mm, 0.85 mm, 0.9 mm, 0.95 mm, 1 mm, 1.05 mm, 1.1 mm, 1.15 mm, 1.2 mm, 1.25 mm, 1.3 mm, 1.35 mm, 1.4 mm, 1.45 mm, or 1.5 mm.

In the tapering flow channel, the width at the narrowest end of the tapering flow channel, which is the end connected to the detection point, can be from about 0.25 mm to 0.5 mm. The width of the narrowest end of the tapering flow channel can be 0.25 mm, 0.3 mm, 0.35 mm, 0.4 mm, 0.45 mm, or 0.5 mm.

A ratio of the width at the widest point to the width at the narrowest point of the tapering flow channel can be from about 2.75-3.25:1. Any value from 2.75-3.25 can be selected in the ratio. A ratio of the width at the widest point to the width at the narrowest point of the tapering flow channel can be about 2.75:1, 2.76:1, 2.77:1, 2.79:1, 2.80:1, 2.81:1, 2.82:1, 2.83:1, 2.84:1, 2.85:1, 2.86:1, 2.87:1, 2.88:1, 2.89:1, 2.90:1, 2.91:1, 2.92:1, 2.93:1, 2.94:1, 2.95:1, 2.96:1, 2.97:1, 2.98:1, 2.99:1, 3.00:1, 3.01:1, 3.02:1, 3.03:1, 3.04:1, 3.05:1, 3.06:1, 3.07:1, 3.08:1, 3.09:1, 3.10:1, 3.11:1, 3.12:1, 3.13:1, 3.14:1, 3.15:1, 3.16:1, 3.17:1, 3.18:1, 3.19:1, 3.20:1, 3.21:1, 3.22:1, 3.23:1, 3.24:1, or 3.25:1.

In the widening flow channel, the width at the narrowest end of the widening flow channel, which is the end connected to the detection point, can be from about 0.25 mm to 0.5 mm. The width of the widest end of the widening flow channel can be 0.25 mm, 0.3 mm, 0.35 mm, 0.4 mm, 0.45 mm, or 0.5 mm.

In the widening flow channel, the width at the widest end of the widening flow channel, which is the end connected to the control point, can be from about 0.75 mm to 1.5 mm. The width of the widest end of the widening flow channel can be about 0.75 mm, 0.8 mm, 0.85 mm, 0.9 mm, 0.95 mm, 1 mm, 1.05 mm, 1.1 mm, 1.15 mm, 1.2 mm, 1.25 mm, 1.3 mm, 1.35 mm, 1.4 mm, 1.45 mm, or 1.5 mm.

A ratio of the width at the widest point to the width at the narrowest point of the widening flow channel can be from about 2.75-3.25:1. Any value from 2.75-3.25 can be selected in the ratio. A ratio of the width at the widest point to the width at the narrowest point of the tapering flow channel can be about 2.75:1, 2.76:1, 2.77:1, 2.79:1, 2.80:1, 2.81:1, 2.82:1, 2.83:1, 2.84:1, 2.85:1, 2.86:1, 2.87:1, 2.88:1, 2.89:1, 2.90:1, 2.91:1, 2.92:1, 2.93:1, 2.94:1, 2.95:1, 2.96:1, 2.97:1, 2.98:1, 2.99:1, 3.00:1, 3.01:1, 3.02:1, 3.03:1, 3.04:1, 3.05:1, 3.06:1, 3.07:1, 3.08:1, 3.09:1, 3.10:1, 3.11:1, 3.12:1, 3.13:1, 3.14:1, 3.15:1, 3.16:1, 3.17:1, 3.18:1, 3.19:1, 3.20:1, 3.21:1, 3.22:1, 3.23:1, 3.24:1, or 3.25:1.

The width of the discharge channel is essentially constant along its length, and can be from about 0.75 mm to 1.5 mm. The width of the discharge channel can be about 0.75 mm, 0.8 mm, 0.85 mm, 0.9 mm, 0.95 mm, 1 mm, 1.05 mm, 1.1 mm, 1.15 mm, 1.2 mm, 1.25 mm, 1.3 mm, 1.35 mm, 1.4 mm, 1.45 mm, or 1.5 mm. The discharge channel provides a fluid connection pathway between the control point and the absorbent pad in the absorbent pad well.

It also was determined that efficiency of the paper-based microfluidic device can be increased by decreasing the length of the flow channels to be 10 mm or less. The length of the tapering flow channel and the widening flow channel can be the same, or the length of the two channels can be different. The length of the tapering flow channel and the widening flow channel independently can be in the range of about 4 to 10 mm. The length of each of the two flow channels independently can be in the range of about 4.0 to 10 mm, or 4.5 to 9.5 mm, or 5.0 to 9.0 mm, or 5.5 to 8.5 mm, or 6.0 mm to 8.0 mm, or 6.5 to 7.5 mm, or 4.5 mm to 7.5 mm. In some configurations, the length of the tapering flow channel and the widening flow channel independently can be about 4.0, 4.25 mm, 4.5 mm, 4.75 mm, 5 mm, 5.25 mm, 5.5 mm, 5.75 mm, 6 mm, 6.25 mm, 6.5 mm, 6.75 mm, 7 mm, 7.25 mm, 7.5 mm, 7.75 mm, 8 mm, 8.25 mm, 8.5 mm, 8.75 mm, 9 mm, 9.25 mm, 9.5 mm, 9.75 mm, or 10 mm. In some configurations, the length of the tapering flow channel and the widening flow channel is the same, and is about 4.5 mm, 4.6 mm, 4.7 mm, 4.8 mm, 4.9 mm, 5.0 mm, 5.1 mm, 5.2 mm, 5.3 mm, 5.4 mm, 5.5 mm, 5.6 mm, 5.7 mm, 5.8 mm, 5.9 mm, 6.0 mm, 6.1 mm, 6.2 mm, 6.3 mm, 6.4 mm, 6.5 mm, 6.6 mm, 6.7 mm, 6.8 mm, 6.9 mm, 7.0 mm, 7.1 mm, 7.2 mm, 7.3 mm, 7.4 mm, or 7.5 mm. In some configurations, the length of the tapering flow channel and the widening flow channel independently is different, and each length can independently be selected from among about 4.5 mm, 4.6 mm, 4.7 mm, 4.8 mm, 4.9 mm, 5.0 mm, 5.1 mm, 5.2 mm, 5.3 mm, 5.4 mm, 5.5 mm, 5.6 mm, 5.7 mm, 5.8 mm, 5.9 mm, 6.0 mm, 6.1 mm, 6.2 mm, 6.3 mm, 6.4 mm, 6.5 mm, 6.6 mm, 6.7 mm, 6.8 mm, 6.9 mm, 7.0 mm, 7.1 mm, 7.2 mm, 7.3 mm, 7.4 mm, and 7.5 mm.

The length of the discharge channel can be in the range of about 4.0 to 10 mm, or 4.25 to 9.5 mm, or 4.5 to 9.0 mm, or 4.75 to 8.5 mm, or 5.0 mm to 8.0 mm. In some configurations, the length of the discharge channel can be about 4.0, 4.25 mm, 4.5 mm, 4.75 mm, 5 mm, 5.25 mm, 5.5 mm, 5.75 mm, 6 mm, 6.25 mm, 6.5 mm, 6.75 mm, 7 mm, 7.25 mm, 7.5 mm, 7.75 mm, 8 mm, 8.25 mm, 8.5 mm, 8.75 mm, 9 mm, 9.25 mm, 9.5 mm, 9.75 mm, or 10 mm.

Each channel has dimensions so that liquid such as blood, serum, urine, and/or water at room temperature (at about 20° C.) can flow from the conjugate pad well to the absorbent pad by capillary forces only. In some configurations, each flow channel along the major part of their length have essentially the same cross-sectional shape although the width might change across the length of the channel.

The test points (the detection point and the control point) can have any shape. The test points can have a shape that is a circle, an oval, an ellipse, a square, a rectangle, a diamond, a parallelogram, a trapezoid, a rhombus, a convex kite, a concave kite, a pentagon, a hexagon, or an octagon. The shape of each test point can be selected independently. For example, one test point can be in the shape of a circle, and one test point can be in the shape of a ellipse. In some configurations, at least one of the test points has a circular shape. In some configurations, both test points have the same shape. In some configurations, each of the detection point and the control point has a circular shape.

The width of the longest bisection of a test point can be independently selected. In some configurations, each detection point and each control point independently can have a width that is about 1.5 times to about 4.4 times the width of the flow path to which it is connected. For example, if a width of the narrowest end of a tapering flow path connected to a detection point, and a width of the narrowest end of the widening flow path connected to a detection point, is about 0.35 mm, the width of the detection point can be in the range of about 0.53 mm to about 1.54 mm. As another example, if a width of the widest end of the widening flow path connected to a control point, and a width of the discharge channel connected to the control point is about 1 mm, the width of the control point can be in the range of about 1.5 mm to about 4.4 mm. By controlling the size of the detection point and the control point, the amount of reagent required can be controlled. When the size of the detection point and the control point is within the recited ranges, the device requires less reagent consumption. In some configurations, the size of the detection point and the control point are different. In some configurations, the size of the detection point and the control point are different. In some configurations, the width of the longest bisection of a detection point and a control point can be independently selected from among about 1 mm, 1.25 mm, 1.50 mm, 1.75 mm, 2 mm, 2.25 mm, 2.50 mm, 2.75 mm, 3 mm, 3.25 mm, 3.50 mm, 3.75 mm, 4 mm, 4.25 mm, 4.50 mm, 4.75 mm, and 5 mm.

The distance between a detection point and a control point in a flow path can be selected to allow separation of the detection signals from the detection point and the control point. In some configurations, the distance between a detection point and a control point in a flow path is about 4 to 8 mm, or 4.25 to 7.75 mm, or 4.5 to 7.5 mm, or 4.75 to 7.25 mm, or 5 to 7 mm, or 5.25 to 6.75 mm, or 5.5 to 6.5 mm, or 5.75 to 6.25 mm, or 5.8 to 6.15 mm, or 5.9 to 6.25 mm. In some configurations, the distance between a detection point and a control point is about 4.0 mm, 4.1 mm, 4.2 mm, 4.3 mm, 4.4 mm, 4.5 mm, 4.6 mm, 4.7 mm, 4.8 mm, 4.9 mm, 5.0 mm, 5.1 mm, 5.2 mm, 5.3 mm, 5.4 mm, 5.5 mm, 5.6 mm, 5.7 mm, 5.8 mm, 5.9 mm, 6.0 mm, 6.1 mm, 6.2 mm, 6.3 mm, 6.4 mm, 6.5 mm, 6.6 mm, 6.7 mm, 6.8 mm, 6.9 mm, 7.0 mm, 7.1 mm, 7.2 mm, 7.3 mm, 7.4 mm, 7.5 mm, 7.6 mm, 7.7 mm, 7.8 mm, 7.9 mm, or 8.0 mm.

The detection point and the control point of the paper-based microfluidic device can include a reagent that is capable of binding to an analyte in a test liquid. Examples of reagents include antibodies, antigens, enzymes, nanoparticles, and conjugates or combinations thereof. In some configurations, a detection point or a control point or both can include two or more reagents. The reagent can, e.g., be applied at detection point or a control point and be allowed to dry, such as it is well known in the art to prepare a microfluidic device with reagents.

The devices provided herein include a conjugate pad well. The conjugate pad well is configured to contain a conjugate pad. The conjugate pad well can have a shape selected from among a circle, an oval, an ellipse, a square, a rectangle, a diamond, a parallelogram, a trapezoid, a rhombus, a convex kite, a concave kite, a pentagon, a hexagon, and an octagon. In some configurations, the conjugate pad well has a circular shape. The diameter or largest cross-sectional length of the conjugate pad well can be in the range of 4-11 mm. In some configurations, the conjugate pad well can be configured to contain a conjugate pad having a diameter or r largest cross-sectional length of about 5-10 mm. The conjugate pad well typically has the same or similar shape as the conjugate pad.

The devices provided herein include an absorbent pad well. The absorbent pad well is configured to contain an absorbent pad. The absorbent pad well can have a shape selected from among a circle, an oval, an ellipse, a square, a rectangle, a diamond, a parallelogram, a trapezoid, a rhombus, a convex kite, a concave kite, a pentagon, a hexagon, and an octagon. In some configurations, the absorbent pad well has a circular shape. The diameter or largest cross-sectional length of the absorbent pad well can be in the range of 4-11 mm. In some configurations, the absorbent pad well can be configured to contain an absorbent pad having a diameter or largest cross-sectional length of about 5-10 mm. The absorbent pads can serve as waste reservoirs, as well as a driving force for capillary flow.

The device provides a fluid flow pathway for a test liquid from the conjugate pad in the conjugate pad well through the tapering flow channel to the detection point, from the detection point through the widening flow channel to the control point, and from the control point through the discharge channel to the absorbent pad in the absorbent pad well. The test liquid can be a biological fluid. The test liquid can be a mixture of a biological fluid and additional components. Exemplary biological fluids include blood and plasma. The biological fluid can be diluted with a solvent, such as water, saline, or phosphate buffered saline, or any other appropriate diluent.

The detection point of the microfluidic device includes a reaction part that includes a material reacting with an analyte or predetermined target molecule or material contained in the biological sample to generate a detectable signal. As an example, the detection point can include capture antibodies fixed at the location of the detection point. The capture antibodies can capture a labelled detection antibody that has formed a complex with a biomarker of interest, such as Cystatin C. In some configurations, the capture antibody captures the labelled detection antibody-cystatin C complex by binding to the cystatin C.

The detectable signal can be, e.g., a fluorescent signal, a colorimetric signal, an electrochemical signal, or a combination thereof. The detectable signal can be from the labelled detection antibody. In one embodiment, the detectable signal is generated by gold nanoparticles conjugated to an antibody to form a labeled detection antibody.

The labelled detection antibodies on, or added to, the conjugate pad can capture the corresponding biomarker (e.g., Cystatin C) from the loading sample to form a complex, and the complex flows through the device from the conjugate pad in the conjugate pad well through the tapering flow channel to the detection point. The fluid containing the complex first encounters a capture antibody (an antibody that binds to the capture antibody) at the detection point where the complex can be captured, while any uncaptured complex or uncomplexed labelled detection antibody passes through and along the widening flow channel to the control point, where either or both bind to an anti-IgG antibody that binds to the IgG of the detection Ab. The detectable signal color comes from a signal generator, such as gold nanoparticles conjugated onto the detection antibody, and their accumulation at the detection point and the control point, respectively, showing the final color intensity, which is the measurable detectable signal. The color signal at the detection point is based on the Cystatin C concentration in the biological sample, with higher concentrations producing stronger signal intensity, e.g., color intensity. The signal, e.g., color signal, at the control point is based on the binding of IgG (from the detection Ab) and an anti-IgG antibody, thus even with no Cystatin C in the loading sample, a signal, e.g., color signal, would be displayed at the control point. The control signal generated at the control point can be used to confirm the assay itself is working properly.

In use, after about 1 minute after loading the sample onto the microfluidic device provided herein, a detectable signal can be measured. For example, after about 1 minute after loading the sample onto the microfluidic device provided herein, a color signal is visually detectable. After about 4-5 minutes of loading the sample onto the microfluidic device, the signal, e.g., a color signal, at the detection point and control point are well defined and by visual inspection appear stable. Washing the channels with buffer removes any unbound floating gold nanoparticles in the channels and at the detection point and the control point that could contribute to the color signal. The channels then are allowed to dry before measuring the color signal. Thus, a stable, consistent, quantitative measurement of the color signal can be taken in about 15 to 20 minutes after loading the sample onto the microfluidic device provided herein.

A detection device can detect, read, analyze, quantitate or any combination of these the detectable signal. For example, the detectable signal can be read by a microscope. The microscope can be a digital microscope. The microscope can be a handheld or portable microscope. The microscope can be a USB microscope. The microscope can be a fluorescent microscope. In some configurations, a portable imaging system can be used. A portable imaging system could simplify detection and can be used in a POC test device. A portable imaging system could include photodiode detector. Advances in camera technologies, such as those now incorporated into many smartphones, also could be configured for use in a portable imaging system for detection of the detectable signal. Any optical readers known in the art can be adapted for use to detect the detectable signal.

In one embodiment, provided is a low-cost paper chip for precise measurement of cystatin C concentration in a drop of blood. Specifically, provided is a paper-based microfluidic device based on a sandwich-lateral flow assay, to measure cystatin C concentration in a blood sample. Loading volume, flow speed and rate are optimized by the unique design of the paper chip (e.g., paper material and channel design). A test point in the paper chip allows optical colorimetric (e.g., converted color intensity) of an antigen-antibody reaction at a defined time point to determine the amount of the cystatin C concentration in the sample.

In comparison to existing enzyme-linked immunosorbent assay (ELISA) kits for measurement of cystatin C in a sample, which require highly trained lab personnel and dedicated equipment such as a plate reader, the paper-based microfluidic device provided herein has the following notable advantages: 1) low cost, 2) easy operation, 3) quantitative and stable measurement, and 4) compatibility for portable and inexpensive optical readers.

Preferred embodiments of the present invention will be described below in more detail with reference to the accompanying drawings. The present invention can, however, be embodied in different forms and should not be construed as limited to the exemplified embodiments set forth herein.

### Fabricating the Device

The paper-based microfluidic device, or paper chip, can be based on a microfluidic sandwich-lateral flow assay. In some configurations, the paper-based microfluidic device can be prepared by printing wax on a substrate having a capillary flow rate of from about 75 to about 120 seconds/4 cm to form the flow channels. The wax defines hydrophobic barriers or a hydrophobic boundary region resulting in microfluidic channels on the hydrophilic paper. The substrate can be any suitable paper. The paper can be a filter paper, a cellulose ester, or a nitrocellulose membrane. Exemplary cellulose ester membranes are sold as a capillary membrane under the Hi-Flow™ Plus brand by MilliporeSigma (Burlington, MA, USA).

In some configurations, a capillary membrane having a flow rate of about 75 seconds/4 cm such as HiFlow™ Plus 75 (HF075, MilliporeSigma, Burlington, MA, USA) can be used. In some configurations, a capillary membrane having a flow rate of about 90 seconds/4 cm such as HiFlow™ Plus 90 (HF090, MilliporeSigma, Burlington, MA, USA) can be used. In some configurations, a capillary membrane having a flow rate of about 120 seconds/4 cm such as HiFlow™ Plus 120 (HF120, MilliporeSigma, Burlington, MA, USA) can be used.

The substrate optionally can be treated with high energy plasma prior to forming the pattern of the microfluidic device on a surface of a substrate. A gas subjected to a high frequency oscillating magnetic field under reduced pressure can ionize the gas molecules resulting in formation of a high energy plasma. The high energy plasma can be used to treat a surface of the substrate. For example, ionized particles in the high energy plasma can combine with surface contaminates and remove such contaminates from the surface by converting them into a gaseous form, such as into methane or carbon dioxide, which can be removed by a vacuum in the plasma device. The high energy plasma particles also can react with a surface to modify a chemical or physical property of the surface. An exemplary high energy plasma generation device that can be used to treat the substrate is a Harrick Plasma Cleaner model PDC-001 (Harrick Plasma, Ithaca, NY). A gas, such as air or oxygen, can be provided to a low pressure reaction chamber of the device (which is under at least some vacuum) and subjected to radio frequency (RF) electromagnetic radiation at 8-12 MHz creating a high energy plasma, at near ambient temperatures, within the chamber. In some embodiments, the room air or oxygen is bled into an evacuated chamber to provide the gas to the chamber, and then subjected to RF radiation until a high energy plasma forms. In some embodiments, a low flowing gas, such as at a rate of 4 to 12 SCFH, can be provided to an evacuated chamber attached to a vacuum pump and subjected to RF radiation until high energy plasma forms, which results in high energy plasma at near ambient temperatures, within the chamber. In the Harrick Plasma Cleaner model PDC-001, the high energy plasma can be observed as a purplish glow.

In an exemplary treatment protocol, the paper substrate is placed in the chamber of a plasma generation device, and then a vacuum pump is activated for a time period of about 2 to 4 minutes to evacuate the air in the chamber of the device. The gas that is selected to be used to generate the high energy plasma (e.g., room air or oxygen) is then slowly introduced into the chamber while under vacuum, and the flow of gas is terminated. Power is then applied to the radio frequency (RF) electromagnetic radiation generator to generate radiation at the desired level (8-12 MHz) and a purplish glow occurs when the high energy plasma has been generated. The sample is subjected to plasma treatment for about 3 to 6 minutes. After treating with the high energy plasma for the desired length of time, the RF radiation is terminated, and the vacuum pump is turned off. Air is allowed to enter the chamber and then the chamber is vented until atmospheric pressure is reached and the door to the chamber of the device can be opened. The high energy plasma-treated substrate then can be used to produce the design of the microfluidic device on the high energy plasma-treated surface of substrate.

The pattern of the microfluidic device on a surface of a substrate can be produced by a traditional solid ink printer by printing a solid wax ink onto a first surface of the substrate. Any solid wax ink known in the art can be used to prepare the patterned solid wax forming the two parallel flow channels on the substrate. The solid wax ink comprises a wax. The solid wax ink can include a wax, a hydrocarbon resin and dyes. The wax can be a fatty amide wax. A colorless solid wax ink also can be used. The printing can be performed using any solid wax ink printer. As an example, the printing can be performed using a ColorQube™ 8570 solid wax ink printer (Xerox, Norwalk, CT, USA).

After the patterned solid wax is formed on the first surface of the substrate, the substrate can be exposed to a temperature above the melting point of the wax contained in the solid wax ink to allow penetration of the wax into the substrate. In some instances, the elevated temperature can be in the range of 60° C.-150° C. The elevated temperature can be 60° C., 65° C., 70° C., 75° C., 80° C., 85° C., 90° C., 95° C., 100° C., 110° C., 115° C., 120° C., 125° C., 130° C., 135° C., 140° C., 145° C., or 150° C. The time for subjecting the substrate with the patterned solid wax to an elevated temperature can be from 1 to 60 seconds. The time for subjecting the substrate with the patterned solid wax to an elevated temperature can be 1, 2, 5, 10, 15, 20, 25, 30, 35, 40, 45, 50, 55, or 60 seconds. The elevated temperature can be achieved by exposing the substrate with the patterned solid wax on a surface thereof to an environment at the targeted elevated temperature, such as by placing the substrate into an oven or heated chamber at the target temperature.

The elevated temperature can be achieved by bringing the reverse side of the substrate (the side of the substrate opposite of the first side of the substrate on which the patterned solid wax is printed) into contact with a heated surface at the targeted elevated temperature. The heated surface at the targeted elevated temperature can be a hot plate, a heated roller, an induction-heated roller, an electrical contact heater, or any direct contact heater known in the art.

After the substrate has been exposed to the elevated temperature for the target period of time, the substrate can be allowed to cool to room temperature, resulting in a substrate that includes a pattern having hydrophobic boundaries. Non-specific binding by the ester cellulose or nitrocellulose in the flow channels can be minimized or prevented by treating the ester cellulose or nitrocellulose with a blocking agent. The blocking agent can include bovine serum albumin (BSA). The BSA can be dissolved in a solvent, such as water, saline, or phosphate-buffered saline (PBS 1×). Treating each channel with a solution containing 0.35 wt % to 0.5 wt % BSA in PBS was found to be effective in blocking or minimizing non-specific binding of the nitrocellulose. Each channel typically is treated with 0.4 wt % BSA in PBS (1×) and dried at room temperature before use. Treatment of the ester cellulose or nitrocellulose with a blocking agent can be done at the time of fabrication of the device, or it can be done prior to use of the device.

Conjugate pads can be made of cellulose fiber, synthetic fiber, or glass fiber. An exemplary glass fiber pad can be made from glass fiber diagnostic pad sheets (available from MilliporeSigma, product GFDX203000, St. Louis, MO, USA). The sheets can be cut to the appropriate size for the microfluid device. In some configurations, the absorbent pads can be cut to be circular in shape and to have a diameter in the range of 5-10 mm. In some configurations, the absorbent pad can be cut to have a circular shape having a diameter of 7 mm. The conjugate pad can be treated with 0.4 wt % BSA in PBS (1×) and dried at room temperature before use.

Absorbent pads can be made of cellulose fiber, synthetic fiber, or glass fiber. An exemplary cellulose fiber pad can be made from C083 cellulose fiber sample pad sheets (available from MilliporeSigma, product CFSP223000, St. Louis, MO, USA). The sheets can be cut to the appropriate size for the microfluid device. In some configurations, the absorbent pads can be cut to be circular in shape and to have a diameter in the range of 5-10 mm. In some configurations, the absorbent pad can be cut to have a circular shape having a diameter of 7 mm.

An exemplary configuration of one embodiment of the paper-based microfluidic device is shown in FIG. 1A. FIG. 1A shows an illustration of plan view of an exemplary paper-based microfluid device 1000. The configuration of the paper-based microfluidic device shown in FIG. 1A includes a first flow channel 100 having a first end and a distal second end, and includes a first detection point T1 and a first control point T2. The illustrated embodiment also includes a second flow channel 200 having a first end and a distal second end, and includes a second detection point T1′ and a second control point T2′.

The first end of the first flow channel 100 and the first end of the second flow channel 200 are connected to bridging flow channel 300. The bridging flow channel 300 also is connected to a conjugate pad well 600, forming a fluid communication path from the conjugate pad well 600 through each of the first flow channel 100 and the second flow channel 200. The distal second end of the first flow channel 100 is connected to a first absorbent pad well 700, so that the first absorbent pad well 700 is in fluid communication with the conjugate pad well 600 via the first flow channel 100. The distal second end of the second flow channel 200 is connected to a second absorbent pad well 705, so that the second absorbent pad well 705 is in fluid communication with the conjugate pad well 600 via the second flow channel 200. First flow channel 100 and second flow channel 200 are essentially parallel.

FIG. 1B shows a schematic plan view of the embodiment of the microfluidic device 1000 depicted in FIG. 1A assembled for use, with a conjugate pad 650 in the conjugate pad well 600, an absorbent pad 750 in absorbent pad well 700, and an absorbent pad 755 in absorbent pad well 705.

Placement of a sample containing an analyte on a conjugate pad 605 located in the conjugate pad well 600 results in capillary flow of the sample containing the analyte through the bridging flow channel 300 and into each of the first flow channel 100 and the second flow channel 200, flowing through the first flow channel 100 to an absorbent pad 750 located in absorbent pad well 700, and flowing through the second flow channel 200 to an absorbent pad 755 located in absorbent pad well 750.

The first flow channel 100 includes a first detection point 400 and a first control point 500. As the test fluid flows through the first flow channel 100, it reaches the first detection point 400 first at a time of T1, and reaches the first control point 500 second, at a time of T2. Accordingly, the first detection point 400 can be referred to as T1 and the first control point 500 can be referred to as T2, particularly in some of the figures herein.

The second flow channel 200 includes a second detection point 405 and a second control point 505. As the test fluid flows through the second flow channel 200, it reaches the second detection point 405 first at a time of T1′, and reaches the second control point 505 second, at a time of T2′. Accordingly, second detection point 405 can be referred to as T1′ and the second control point 505 can be referred to as T2′, particularly in some of the figures herein.

In some configurations, the time T1 is about or essentially the same as the time T1′, and the time T2 is about or essentially the same as the time T2′.

A capture material that reacts with cystatin C analyte contained in the test biological fluid can be included in each of the first detection point 400 and the second detection point 405 to capture and allow quantification of cystatin C in the test fluid. For example, a cystatin C capture antibody can be applied to each of the first detection point 400 and the second detection point 405 and allowed to dry overnight prior to use of the microfluidic device.

A reaction material also can be included in the first control point 500 and the second control point 505 to generate a second detectable signal. The reaction material included in the first control point 500 and the second control point 505 can be a material that interacts with capture antibody that did not react with the cystatin C analyte. In some embodiments, the reaction material included in the first control point 500 and the second control point 505 can be an antibody that binds to the capture antibody. For example, when the capture antibody is an IgG antibody, a goat anti-mouse IgG antibody can be applied to each of the first control point 500 and the second control point 505 and allowed to dry overnight prior to use of the microfluidic device.

An exemplary configuration of another embodiment of the paper-based microfluidic device is shown in FIG. 7A. FIG. 7A shows an illustration of plan view of an exemplary paper-based microfluid device 2000. The configuration of the paper-based microfluidic device 2000 shown in FIG. 7A includes a conjugate pad well 2600. Attached to conjugate pad well 2600 is a tapering flow channel 2350. Tapering flow channel 2350 has a wide first end and a narrow distal second end. Tapering flow channel 2350 is attached via its wide first end to conjugate pad well 2600. The narrow distal second end of tapering flow channel 2350 is attached to detection point 2400. Tapering flow channel 2350 provides a fluid flow path from the conjugate pad well 2600 to detection point 2400 so that detection point 2400 is in fluid communication with conjugate pad well 2600. The device also includes a widening flow channel 2450. Widening flow channel 2450 has a narrow first end and a wide distal second end. Widening flow channel 2450 is attached via its narrow first end to detection point 2400. The wide distal second end of widening flow channel 2450 is attached to control point 2500. Widening flow channel 2450 provides a fluid flow path from the detection point 2400 to control point 2500 so that detection point 2400 is in fluid communication with detection point 2400. The device also includes a discharge channel 2550 that connects the control point 2500 to an absorbent pad well 2700. Discharge channel 2550 can have a constant width from the control point 2500 to the absorbent pad well 2700, or the width of discharge channel 2550 can increase as it approaches absorbent pad well 2700. Discharge channel 2550 provides a fluid flow path from the control point 2500 to absorbent pad well 2700 so that control point 2500 is in fluid communication with absorbent pad well 2700.

FIG. 7B shows a schematic plan view of the embodiment of the microfluidic device 2000 depicted in FIG. 7A assembled for use, with a conjugate pad 2650 in the conjugate pad well 2600, an absorbent pad 2750 in absorbent pad well 2700.

Placement of a sample, such as a test biological fluid, containing an analyte on a conjugate pad 2650 located in the conjugate pad well 2600 results in capillary flow of the sample containing the analyte through the tapering flow channel 2350 to detection point 2400.

A capture material that reacts with cystatin C analyte contained in the test biological fluid can be included in each of the detection point 2400 to capture and allow quantification of cystatin C in the test fluid. For example, a cystatin C capture antibody can be applied to detection point 2400 and allowed to dry overnight prior to use of the microfluidic device.

A reaction material also can be included in the control point 2500 to generate a second detectable signal. The reaction material included in the control point 2500 can be a material that interacts with capture antibody that did not react with the cystatin C analyte. In some embodiments, the reaction material included in control point 2500 can be an antibody that binds to the cystatin C capture antibody. For example, when the capture antibody is an IgG antibody, a goat anti-mouse IgG antibody can be applied to control point 2500 and allowed to dry overnight prior to use of the microfluidic device.

### Methods of Use

For use, the flow channels 100 and 200 and bridging flow channel 300 of the first exemplary device, or the tapering flow channel and widening flow channel of the second exemplary device, are each treated with 0.4 wt % BSA in PBS (1×) and allowed to dry at room temperature for 1 hour. The channels then are rinsed with 0.1 wt % Tween 20 (polyoxyethylene sorbitol ester) in PBS (1×) and the membrane was allowed to dry at room temperature for approximately 30 minutes. Treatment with the surfactant helps to modulate the surface tension of the fluid as it flows in the flow channels.

A conjugate pad is placed into the conjugate pad well, and an absorbent pad is placed in the absorbent pad well(s).

A detection material is placed on the conjugate pad, followed by loading a sample of the test fluid containing the cystatin C analyte on the conjugate pad. The detection material can be an antibody that reacts with the cystatin C analyte. The detection antibody can be a conjugate that includes a fluorescent material, a phosphor, a nanogold particle, or a combination thereof. In one embodiment, a cystatin C detection antibody can be the detection material, and the test fluid can be blood or a blood fraction. The cystatin C detection antibody can be a gold nanoparticle-labeled detection antibody. The detection antibody will react with any cystatin C in the sample. The loading of the detection antibody and the sample forms a test fluid that wets the conjugate pad adhering it to the conjugate pad well and creating a fluid connection with the flow paths.

For the embodiment show as microfluidic device 1000 in FIGS. 1A and 1B, a time of a few minutes is allowed to pass to allow the test fluid to flow via capillary action from the conjugate pad 650 in conjugate pad well 600 to and through bridging flow channel 300 to and through the first flow channel 100 and second flow channel 200, and ultimately into absorbent pad 750 in absorbent pad well 700, and into absorbent pad 755 in absorbent pad well 705, respectively. An aliquot of PBS (1×) then can be placed on the conjugate pad 650 for post-washing. The membrane substrate of the device can be allowed to dry at room temperature before taking images to detect and quantify the detectable signal.

For the embodiment show as microfluidic device 2000 in FIG. 7A and FIG. 7B, a time of a few minutes is allowed to pass to allow the test fluid to flow via capillary action from the conjugate pad 2650 in conjugate pad well 2600 through tapering flow channel 2350, through detection point 2400, through widening flow channel 2450 to control point 2500, and through discharge channel 2500 to absorbent pad 2750 in absorbent pad well 2700. An aliquot of PBS (1×) then can be placed on the conjugate pad 2650 for post-washing. The membrane substrate of the device can be allowed to dry at room temperature before taking images to detect and quantify the detectable signal.

Any imaging device known in the art can be used for image capture. For example, a portable electronic USB microscope (Dino-Lite USB microscope, Cat. #AD4113T, available from Dunwell Tech, Inc., Torrance, CA, USA) can be used to detect the detectable signal. The paper-based microfluidic device can be placed on a stage under the Dino-lite USB Microscope, and different magnifications (e.g., 20×, 30×, or 40×) of the USB microscope can be viewed and the magnification providing the optimum focus of the detection point(s) and control point(s) of the paper-based microfluidic device selected. Appropriate software for image capture and image analysis can be used. Exemplary software for image capture is DinoCapture 2.0 (Version 1.5.41) software. The DinoCapture 2.0 (Version 1.5.41) software with the default settings can be used for acquiring the images, and the “Snapshot” function of the software can be used for recording the color signal that displayed on the paper device.

The color signal then can be analyzed using any image analysis software known in the art can be used to evaluate the captured images. Exemplary image analysis software that can be used to analyze the color images captured by the USB microscope is ImageJ software, an image processing program developed at the National Institutes of Health and the Laboratory for Optical and Computational Instrumentation.

A color image with 1280×1024 pixels that covers the detection point area or that covers the control point area can be captured for color signal measurement. One area of interest (AOI) can be selected using the “Oval Selections” function from ImageJ for each detection point (i.e., top and bottom T1 points) of the device. The color of each AOI can be measured and inverted using “RGB Measure” function from ImageJ, and then can be split into multiple RGB channels, from which the “(R+G+B)/3” channel can be used to calculate the signal intensity. The mean value of “(R+G+B)/3” channel can be recorded and deducted by 255 for the color intensity value of each AOI. Then the average of the two AOI values obtained from top and bottom T1 points can be used as the final color intensity value for each concentration.

The final color intensity values from different concentrations (i.e., 0, 0.5, 1, 2, 4, and 8 mg/L) of cystatin C standard samples can be used to create a calibration curve using OriginPro software (OriginLab Corporation, Northampton, MA, USA). The “Linear Fit” function of the OriginPro software then can be applied to check the linearity and limit of detection (LOD).

In use, cystatin C in the sample placed on the conjugate pad will react with the gold nanoparticle-labeled detection antibody in the conjugation pad. In the microfluidic device 1000 shown in FIGS. 1A and 1B, the resulting conjugate will move through the flow channels until it reaches detection points 400 and 405, where it will react with the capture antibody at detection points 400 and 405 to form a sandwich of (gold nanoparticle-labeled detection antibody)-(analyte)-(capture antibody). Any detection antibody that did not react with the cystatin C analyte will pass through detection points 400 and 405 and be captured by the reaction material at control points 500 and 505, which can be, e.g., a goat anti-mouse IgG antibody that can capture the gold nanoparticle-labeled detection antibody that did not form a conjugate with the analyte. In the microfluidic device 2000 shown in FIGS. 7A and 7B, the resulting conjugate will move through tapering flow channel 2350 until it reaches detection point 2400, where it will react with the capture antibody at detection points 2400 to form a sandwich of (gold nanoparticle-labeled detection antibody)-(analyte)-(capture antibody). Any detection antibody that did not react with the cystatin C analyte will pass through detection point 2400 through widening flow channel 2450 and be captured by the reaction material at control point 2500, which can be, e.g., a goat anti-mouse IgG antibody that can capture the gold nanoparticle-labeled detection antibody that did not form a conjugate with the analyte.

Different concentrations of cystatin-C can be prepared and added to a control test sample in order to generate a correlation between the detection signal observed and the concentration of analyte in the sample. There will be a positive correlation between the intensity of the generated signal and the analyte content in the sample, which can be used to quantify the analytes present in the sample. For example, recombinant human cystatin C (HyTest Ltd., Cat. #8CY5) can be reconstituted with Phosphate Buffered Saline (PBS, 1×) to the stock concentration at 1 mg/mL, followed by secondary dilution with PBS (1×) to prepare different concentrations of cystatin C. As will be appreciated by one of skill in the art, a sample of interest can be diluted to a suitable concentration so as to fall within the detection range.

Other suitable reagents for use as part of a detectable reaction for the target cystatin C analyte will be readily apparent to one of skill in the art. For example, any suitable reagent used in a commercially available kit for detection of cystatin C can be used within the paper-based microfluid device provided herein. That is, there are a large number of assays known in the art which produce a detectable reaction, all of which can be used in the paper-based microfluidic device provided herein. An advantage of the device provided herein is that significantly lower amounts of each reagent than used in traditional ELISA assays is required. Similarly, the conditions under which these reactions can be detected are also well-known in the art and can be used with the paper-based microfluid device provided herein and the methods of detecting an analyte, such as cystatin C, using the device provided herein.

As will be appreciated by one of skill in the art, the method of the invention may be used to monitor kidney damage of an individual, as a means of monitoring disease progression. The method of the invention may also be used for screening at-risk individuals, for example, individuals with a familial history of chronic kidney disease, with diabetes mellitus, high blood pressure or glomerulonephritis.

Individuals diagnosed with chronic kidney disease and who show signs of the disease worsening or anyone who is being monitored regularly and shows signs of the disease worsening may be prescribed medication to reduce blood pressure or may be assigned kidney protective treatments such as aggressive BP lowering, Renin Angiotensin Aldosterone System Inhibitors (RAASi), Sodium Glucose Co-transporter-2 inhibitors (SGLT2i), or any combination thereof. The Cystatin C based eGFR (estimated glomerular filtration rate) method makes feasible more frequent and even daily monitoring of kidney status in patients at high risk either of AKI (acute kidney injury), or of acute or chronic deterioration requiring dialysis initiation, a strategy which could help avoid unplanned emergency room visits or emergency dialysis starts.

### EXAMPLES

The invention will now be further explained by way of examples. The following examples are included for illustrative purposes only and are not intended to limit the scope of the embodiments provided herein.

### Example 1. Comparing Paper-Based Microfluid Device Designs

The design of one embodiment of the paper-based microfluidic device provided herein was compared to another paper-based microfluidic device design to demonstrate improvements achieved.

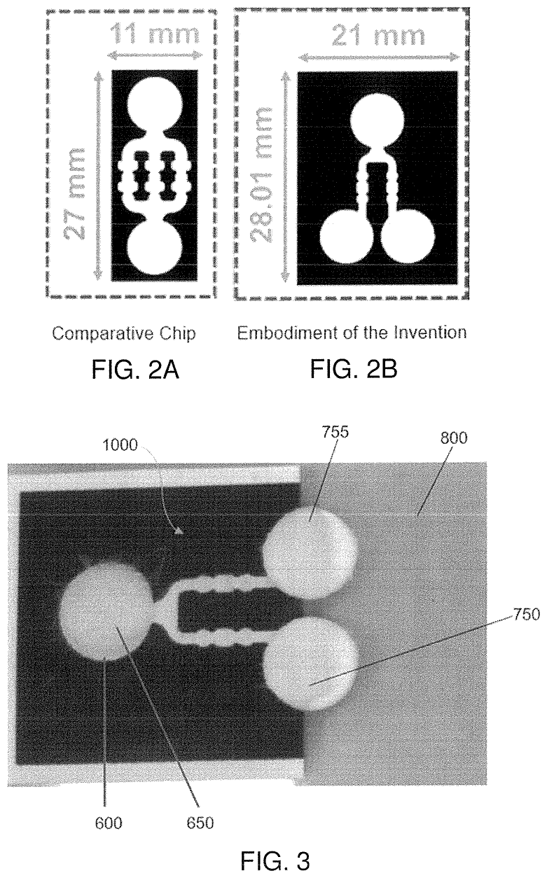

The design differences can be seen when comparing FIG. 2A to FIG. 2B. As can be seen in FIG. 2B, the paper-based microfluidic device provided herein has a more compact design with respect to the width and length of the flow channels, compared to the comparative paper chip depicted in FIG. 2A. The embodiment of the invention depicted in FIG. 2B was designed to have one conjugate pad, similar to prior designs, but was modified to include only two flow channels instead of the three flow channels depicted in the device of FIG. 2A. The width of each the flow paths in the paper-based microfluidic device provided herein shown in FIG. 2B also was reduced, e.g., from 1.5 mm to 1 mm, which resulted in reduced flow rate compared to previous designs. It also was determined that this reduction in flow rate improved the binding efficiency of different reagents at T1 and T2 points, which resulted in improved signal generation. It also was determined that the lengths of the flow channels could be reduced with no effect on color signal generation or intensity. Finally, it was determined that including two separate absorbent pads, one separately attached to each flow channel, increased the solution absorbent capacity.

### Example 2. Fabrication of the Paper Chips

Exemplary paper-based microfluidic chips were produced by solid wax ink printing the design on a cellulose ester substrate. Each microfluidic device pattern was designed in SolidWorks®, and then printed on a HiFlow Plus 90 membrane (MilliporeSigma, Cat. #HF09004XSS) using a solid wax ink printer (ColorQube™ 8570, Xerox, Norwalk, CT, USA). The membrane with the printed wax pattern was then heated on a hotplate at 125° C. for 30 seconds to melt the wax ink. The melted wax ink penetrated into the HiFlow Plus 90 membrane and formed hydrophobic boundaries. Round 7 mm-diameter conjugate pads were prepared from glass fiber (glass fiber conjugate pad, MilliporeSigma, Cat. #GFDX203000). The conjugate pads were treated with 0.4% BSA in PBS (1×), and dried at room temperature, before use. The conjugate pad fits in the conjugate pad well of the device. Round 7 mm-diameter absorbent pads were made from cellulose fiber (cellulose fiber sample pad, MilliporeSigma, Cat. #CFSP223000). The absorbent pads fit in the absorbent pad wells of the device.

In some embodiments, the microfluidic device is modified to cut away a portion of the absorbent pad wells, the modified microfluidic device is applied to a substrate having an adhesive surface, and the absorbent pads are placed in the modified absorbent pad wells, affixed in place by the adhesive on the surface of the substrate. An illustration is shown in FIG. 3. In FIG. 3, a substrate 800 that has an adhesive surface is provided. A portion of the absorbent wells of the microfluidic device 1000 are removed, the modified microfluidic device is placed in the adhesive surface of substrate 800, and then absorbent pad 750 is placed in the remaining absorbent pad well 500 (not visible in the photo), and absorbent pad 755 is placed in the remaining absorbent pad well 505 (not visible in the photo), fixed into place by the adhesive surface of substrate 800. Conjugate pad 650 is placed in conjugate pad well 600.

### Example 3. Performing an Analysis of Cystatin C Test Samples

Tests with Cystatin C capture Ab were performed to validate the paper-based microfluidic device. Gold nanoparticle-conjugated antibodies (AuNPs-Ab) to cystatin C were prepared. A cystatin C detection antibody (HyTest, Cat, No. Cyst28) was conjugated with gold nanoparticles according to the manufacturer's protocol of Gold Conjugation Kit (Abcam, Cat. No. ab188215). Recombinant human cystatin C (HyTest Ltd., Cat. No. 8CY5) was reconstituted with phosphate buffered saline (PBS, 1×) to the stock concentration of 1 mg/ml, followed by secondary dilution with PBS (1×) to the desired concentrations.

A 0.05 μL aliquot of Cystatin C capture Ab (3 mg/mL) was loaded at T1 and T1′ points of the device. A 0.05 μL aliquot of goat anti-mouse IgG (H+L, ThermoFisher Scientific, Cat. No. 31160, 2.32 mg/mL) was loaded at T2 and T2′ points of the membrane. The device was allowed to dry at room temperature (about 20° C.) for overnight. The flow channels of the devices then were blocked with 20 μL of 0.4% BSA in PBS (1×) and the device was allowed to dry at room temperature for 1 h. The membrane then was rinsed with 20 μL of 0.1% Tween in PBS (1×) 1 time, and the membrane was allowed to dry at room temperature (approximately 30 minutes). Then, 2 μL AuNPs-Ab (the gold nanoparticle-labelled cystatin C detection antibody) of a 50 μg/mL solution of the gold nanoparticle-labelled cystatin C detection antibody was loaded on the pretreated conjugate pad (i.e., the conjugate pad that was treated with 0.4% BSA in PBS (1×)), followed by loading 20 μL of sample on the conjugate pad. After a few minutes, the fluid traversed the length of the flow paths. Then, a 20 μL aliquot of PBS (1×) was loaded on the conjugate pad for post-washing, and after the washing, the membrane was allowed to dry at room temperature before taking images.

To acquire the images, the paper-based microfluidic device was placed on a stage under a Dino-lite USB Microscope (Cat. #AD4113T), and different magnifications (e.g., 20×, 30×, or 40×) of the USB microscope were adjusted to determine the best focus. DinoCapture 2.0 (Version 1.5.41) software with the default settings were applied for taking images, and the “Snapshot” function of software was used for recording the color signal that displayed on the paper device. The color images captured by the USB microscope were then analyzed using ImageJ software.

A color image with 1280×1024 pixels that covers the detection areas was captured for color signal measurement. One area of interest (AOI) was selected using the “Oval Selections” function from ImageJ for each detection point (e.g., top and bottom T1 points) of the device. The color of each AOI was measured and inverted using “RGB Measure” function from ImageJ, and then split into multiple RGB channels, from which the “(R+G+B)/3” channel was used to calculate the signal intensity. The mean value of “(R+G+B)/3” channel was recorded and deducted by 255 for the color intensity value of each AOI. Then the average of the two AOI values obtained from top and bottom T1 points was used as the final color intensity value for each concentration. The final color intensity values from different concentrations (i.e., 0, 0.5, 1, 2, 4, and 8 mg/L) of Cystatin C standard samples were used to create the calibration curve using OriginPro software. “Linear Fit” function from OriginPro was then applied to check the linearity and limit of detection (LOD).

### Example 4. Color Signal Display

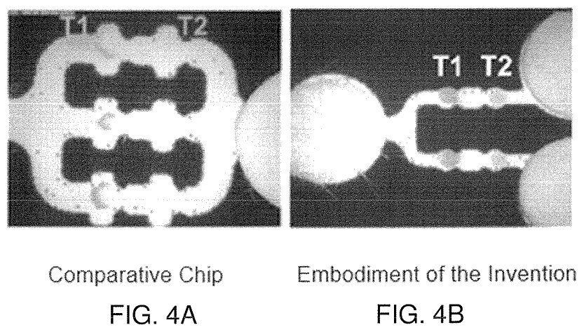

The color signal observed for the paper-based microfluidic device provided herein is illustrated in FIG. 4B, and a comparative paper chip is illustrated in FIG. 4A. Compared to the comparative chip shown in FIG. 4A, the embodiment of the paper-based microfluidic device provided herein shown in FIG. 4B exhibits significantly improved color signal intensity and display. The color signal of the paper-based microfluidic device provided herein is more uniformly displayed at the test points (T1 and T2) than the color signal achieved in a comparative paper chip shown in FIG. 4A.

### Example 5. Calibration Curve and Limit of Detection (LOD)

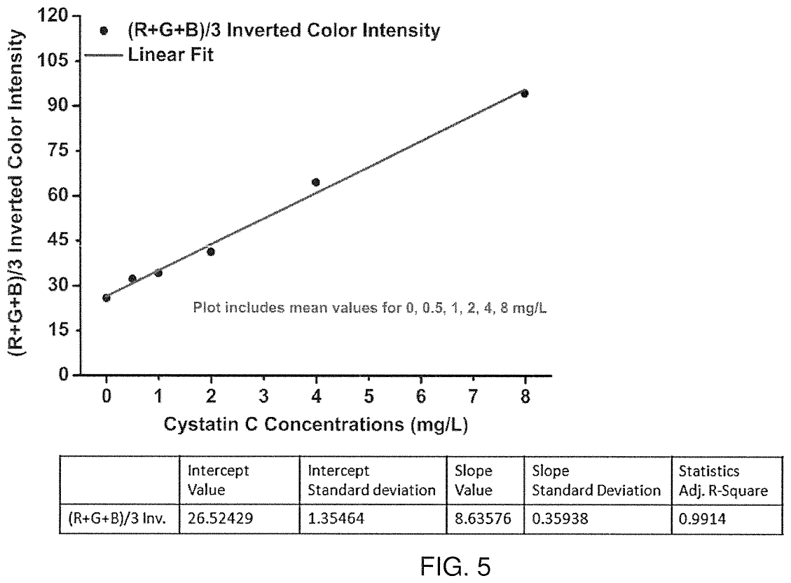

To produce a calibration curve for the cystatin-C detection, a cystatin-C standard was serially diluted to different concentrations (0.5 to 8 mg/L and blank). The detectable signal was captured after loading the standard on the paper-based microfluidic chip provided herein. The calibration curve was plotted after subtracting the blank signal. The results are shown in FIG. 5.

As can be seen from the results, the paper-based microfluidic chip provided herein with the design shown in FIG. 1A achieved an R2 value of 0.9914 and LOD of 0.47 mg/L. This LOD is well fit for the detection threshold (1 mg/L) of cystatin C concentration in a serum/plasma sample for indicating the early stage of chronic kidney disease.

### Example 6. Validation of the Paper-Based Microfluidic Chip Using CKD Samples

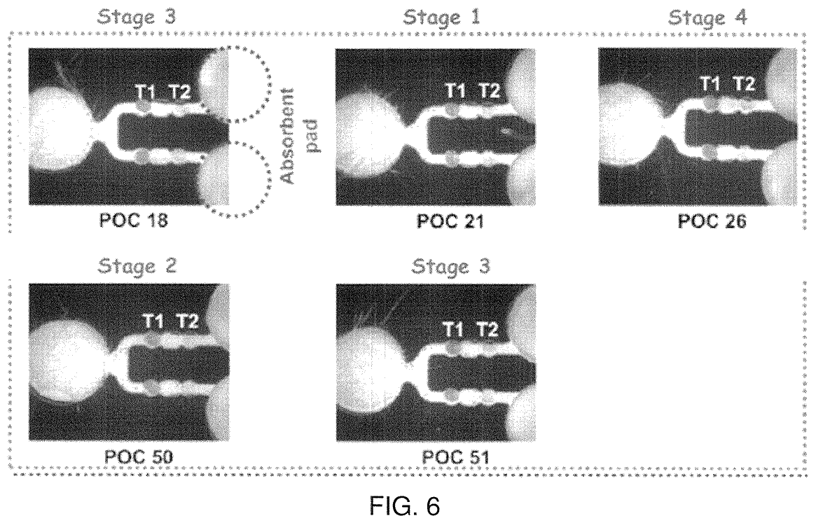

The paper-based microfluid chip provided herein was further validated using blood samples from patients who have been diagnosed with CKD. The collected patient information includes disease stage, gender, and some biomarker measurements. The cystatin-C concentration in the samples using the paper-based microfluidic chip provided herein was measured. The results are shown in FIG. 6.

The data demonstrate that the color signal is clearly visible and the signal intensity generally agrees with the disease stage of the patient (Stage 1 through Stage 4). The color signal was determined to generally be too high if calculated based on the corresponding cystatin C concentration based on the calibration curve comparing to the expected level. This discrepancy has been attributed to the age and storage conditions of the samples. The tested plasma samples had been stored in a deep freezer for many years, which resulted in a high level of non-specific binding on the paper chip. The same samples were tested by traditional ELISA after 1000× dilution and the results are reasonable (in this case, the non-specific binding was significantly reduced by dilution).

### Example 7. Comparing Paper-Based Microfluid Device Designs

The design of another embodiment of the paper-based microfluidic device provided herein was compared to a different paper-based microfluidic device design to demonstrate improvements achieved.

The design differences can be seen when comparing FIG. 2A to FIG. 7A. As can be seen in FIG. 2B, the embodiment of the invention depicted in FIG. 7A was designed to have one conjugate pad and one absorbent pad, similar to prior designs, but was modified to include only one flow channel instead of the three flow channels depicted in the device of FIG. 2A. The device shown in FIG. 7A includes a combination of a tapering flow channel 2350 and a widening flow channel 2450. The tapering flow channel 2350 has a wide first end and a narrow distal second end, and the tapering flow channel 2350 is attached via its wide first end to the conjugate pad well 2600. The narrow distal second end of tapering flow channel 2350 is connected to a detection point 2400. A widening flow channel 2450 has a narrow first end and a wide distal second end, and the narrow first end is attached to the detection point 2400. The wide distal second end of the widening flow channel 2450 is attached to a control point 2500, which is attached to a first end of a discharge channel 2550. The second end of the discharge channel is attached to an absorbent pad well 2700.

Varying channel width was determined to allow better control the fluid flow and thus antigen capture by the antibody at the test point. Because of the narrowing width of the tapering flow channel as it goes from the conjugate pad well to the detection point, the flow of fluid containing antigen slows down as it approaches the detection point due to the narrowing channel. This reduction in flow rate improved the binding efficiency of different reagents at the detection point, which resulted in improved signal generation. Because of the increasing width of the widening flow channel as is goes from the detection point to the control point, fluid flow speeds up once passed the detection point.

### Example 8. Fabrication of the Paper Chips

The exemplary embodiment of the paper-based microfluidic chi shown in FIG. 7A was produced by solid wax ink printing the design on a cellulose ester substrate that was surface-treated with plasma. Surface-treating the cellulose ester substrate was found to improve antibody immobilization. A HiFlow™ Plus 90 membrane (HF090, MilliporeSigma, Burlington, MA, USA) to be used as the paper substrate was placed in the chamber a bench top plasma cleaner (Harrick Plasma Cleaner Model PDC-001, Harrick Scientific, Ithaca, NY) and the chamber was closed. Vacuum was applied to the chamber for 2-3 minutes, and the room air was introduced into the chamber through a needle valve followed by closing the needle valve. The device was set to high power mode, which applied about 29 W to an RF (radio frequency) coil to generate an RF frequency of 8-12 MHz, generating a plasma at near ambient temperatures inside the chamber. A purplish glow was observed within the chamber, which was a visual confirmation that plasma was present. Plasma treatment was done for a time of about 4 to 5 minutes. The RF level was then set to off, the plasma cleaner was turned off, the vacuum pump was turned off, and the needle valve was opened to allow the chamber to come to atmospheric pressure to allow the door to be opened to retrieve the plasma-treated substrate.

The design shown in FIG. 7A was designed in SolidWorks®. An exemplary embodiment had the following dimensions. The conjugate pad well and the absorbent pad well each had a circular shape, with a diameter of 7 mm. The detection point and the control point each had a circular shape with a diameter of 1.5 mm. The tapering flow channel had a wide end with a width of 1 mm, and a narrow end with a width of 0.35 mm, and the length of the tapering flow channel was 5.8 mm. The widening flow channel had a width at its narrow end of 0.35 mm and a width at its wide end of 1 mm, and the length of the widening flow channel was 5.9 mm. The discharge channel had a width at each end of 1 mm, and the length of the discharge channel was 4.5 mm. The design was then printed on the surface of the plasma-treated substrate using a solid wax ink printer (ColorQube™ 8570, Xerox, Norwalk, CT, USA). The plasma-treated substrate with the printed wax pattern was then heated on a hotplate at 125° C. for 30 seconds to melt the wax ink. The melted wax ink penetrated into the plasma-treated HiFlow Plus 90 membrane and formed hydrophobic boundaries. Round 7 mm-diameter conjugate pads were prepared from glass fiber (glass fiber conjugate pad, MilliporeSigma, Cat. #GFDX203000). The conjugate pads were treated with 0.4% BSA in PBS (1×), and dried at room temperature, before use. The conjugate pad fits in the conjugate pad well of the device. Round 7 mm-diameter absorbent pads were made from cellulose fiber (cellulose fiber sample pad, MilliporeSigma, Cat. #CFSP223000). The absorbent pads fit in the absorbent pad wells of the device.

In some embodiments, the microfluidic device was modified to cut away a portion of the absorbent pad well, and the modified microfluidic device was applied to a substrate having an adhesive surface, and an absorbent pad was placed in the modified absorbent pad well, affixed in place by the adhesive on the surface of the substrate.

### Example 9. Performing an Analysis of Cystatin C Test Samples

Tests with Cystatin C capture Ab were performed to validate the paper-based microfluidic device. Gold nanoparticle-conjugated antibodies (AuNPs-Ab) to cystatin C were prepared. A cystatin C detection antibody (HyTest, Cat, No. Cyst28, an IgG antibody) was conjugated with gold nanoparticles according to the manufacturer's protocol of Gold Conjugation Kit (Abcam, Cat. No. ab188215). Recombinant human cystatin C (HyTest Ltd., Cat. No. 8CY5) was reconstituted with phosphate buffered saline (PBS, 1×) to the stock concentration of 1 mg/ml, followed by secondary dilution with PBS (1×) to the desired concentrations.

A 0.05 μL aliquot of Cystatin C capture Ab (3 mg/mL) was loaded at the detection point of the device. A 0.05 μL aliquot of goat anti-mouse IgG (H+L, ThermoFisher Scientific, Cat. No. 31160, 2.32 mg/mL) was loaded at the control point of the membrane. The device was allowed to dry at room temperature (about 20° C.) for overnight. The flow channels of the devices then were blocked with 20 μL of 0.4% BSA in PBS (1×) and the device was allowed to dry at room temperature for 1 h. The membrane then was rinsed with 20 μL of 0.1% Tween in PBS (1×) 1 time, and the membrane was allowed to dry at room temperature (approximately 30 minutes). Then, 2 μL AuNPs-Ab (the gold nanoparticle-labelled cystatin C detection antibody) of a 50 μg/mL solution of the gold nanoparticle-labelled cystatin C detection antibody was loaded on the pretreated conjugate pad (i.e., the conjugate pad that was treated with 0.4% BSA in PBS (1×)), followed by loading 20 μL of sample on the conjugate pad. After a few minutes, the fluid traversed the length of the flow paths. Then, a 20 μL aliquot of PBS (1×) was loaded on the conjugate pad for post-washing, and after the washing, the membrane was allowed to dry at room temperature before taking images.

To acquire the images, the paper-based microfluidic device was placed on a stage under a Dino-lite USB Microscope (Cat. #AD4113T), and different magnifications (e.g., 20×, 30×, or 40×) of the USB microscope were adjusted to determine the best focus. DinoCapture 2.0 (Version 1.5.41) software with the default settings were applied for taking images, and the “Snapshot” function of software was used for recording the color signal that displayed on the paper device. The color images captured by the USB microscope were then analyzed using ImageJ software.

A color image with 1280×1024 pixels that covers the detection areas was captured for color signal measurement. One area of interest (AOI) was selected using the “Oval Selections” function from ImageJ for each detection point (e.g., top and bottom T1 points) of the device. The color of each AOI was measured and inverted using “RGB Measure” function from ImageJ, and then split into multiple RGB channels, from which the “(R+G+B)/3” channel was used to calculate the signal intensity. The mean value of “(R+G+B)/3” channel was recorded and deducted by 255 for the color intensity value of each AOI. Then the average of the two AOI values obtained from top and bottom T1 points was used as the final color intensity value for each concentration. The final color intensity values from different concentrations (i.e., 0, 0.5, 1, 2, 4, and 8 mg/L) of Cystatin C standard samples were used to create the calibration curve using OriginPro software. “Linear Fit” function from OriginPro was then applied to check the linearity and limit of detection (LOD).

### Example 10. Color Signal Display

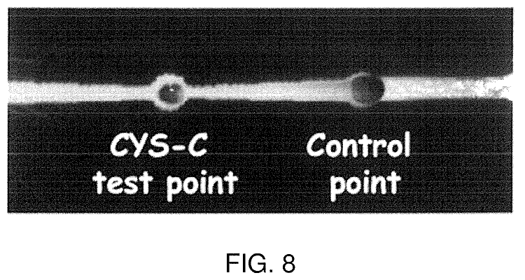

The color signal observed for the embodiment of the paper-based microfluidic device depicted in FIG. 7A is illustrated in FIG. 8. The device exhibited significantly improved color signal intensity and display, with a uniform color signal.

### Example 11. Calibration Curve and Limit of Detection (LOD)

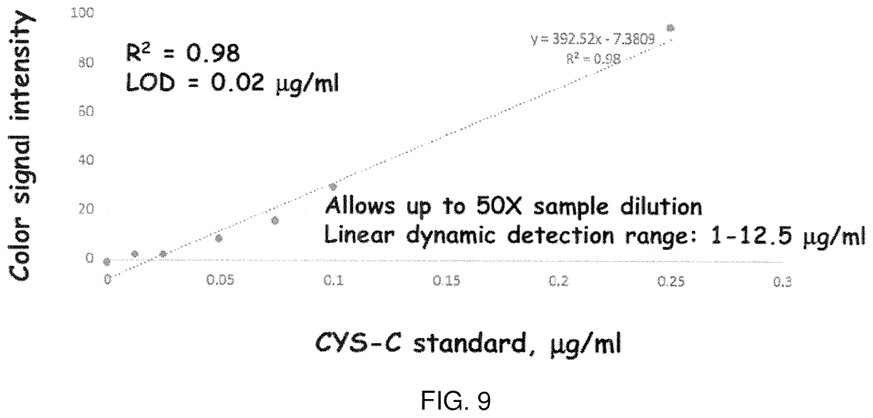

To produce a calibration curve for the cystatin-C detection, a cystatin-C standard was serially diluted to different concentrations (0.05 to 0.3 μg/L and blank). The detectable signal was captured after loading the standard on the paper-based microfluidic chip shown in FIG. 7B. The calibration curve was plotted after subtracting the blank signal. The results are shown in FIG. 9.

As can be seen from the results, the paper-based microfluidic chip provided herein with the design shown in FIG. 7A achieved an R2 value of 0.98 and level of detection (LOD) of 0.02 μg/L. This LOD is well fit for the detection threshold (1 mg/L) of cystatin C concentration in a serum/plasma sample for indicating the early stage of chronic kidney disease. This embodiment of the paper-based microfluid chip provided herein demonstrated a linear dynamic detection range of 1-12.5 μg/mL, and improved LOD down to 20 ng/ml, which allows up to 50× sample dilution while maintaining the clinically relevant linear detection range. The 50× sample dilution is expected to effectively resolve any high signal background issue that can occur due to the sample matrix effect.

### LISTING OF FIGURE REFERENCE NUMBERS

100 first flow channel
200 second flow channel
300 bridging flow channel
400 first detection point (T1)—first flow channel
405 second detection point (T1′)—second flow channel
500 first control point (T2)—first flow channel
505 second control point (T2′)—second flow channel
600 conjugate pad well
650 conjugate pad
700 first absorbent pad well connected to first flow channel
705 second absorbent pad well connected to second flow channel
750 first absorbent pad
755 second absorbent pad
800 substrate having an adhesive surface
1000 one embodiment of the paper-based microfluid device
2000 a second embodiment of the paper-based microfluid device
2350 tapering flow channel
2400 detection point
2450 widening flow channel
2500 control point
2550 discharge channel
2600 conjugate pad well
2650 conjugate pad
2700 absorbent pad well
2750 absorbent pad

1.-49. (canceled)

50. A paper-based microfluidic device, comprising:
a paper-based substrate; and
on a surface of the paper-based substrate:
a conjugate pad well;
a tapering flow channel having a wide first end and a narrow distal second end, the tapering flow channel attached via its wide first end to the conjugate pad well;
a detection point attached to the narrow distal second end of the tapering flow channel;
a widening flow channel having a narrow first end and a wide distal second end, the narrow first end attached to the detection point;
a control point attached to the wide distal second end of the widening flow channel;
a discharge channel having a first end and a second end, and attached via the first end to the control point; and
an absorbent pad well attached to the second end of the discharge channel.

51. The microfluidic device of claim 50, wherein the conjugate pad well, the tapering flow channel, the detection point, the widening flow channel, the control point, the discharge channel, and the absorbent pad well are defined by a hydrophobic material forming a hydrophobic boundary region on the paper-based substrate.

52. (canceled)

53. (canceled)

54. The microfluidic device of claim 50, wherein a surface of the paper-based substrate has been treated with a high energy plasma.

55. The microfluidic device of claim 50, wherein:
the conjugate pad well has a shape selected from the group consisting of a circle, an oval, an ellipse, a square, a rectangle, a diamond, a parallelogram, a trapezoid, a rhombus, a convex kite, a concave kite, a pentagon, a hexagon, and an octagon, and a diameter or largest cross-sectional length that is in the range of 4-11 mm; and
the absorbent pad well has a shape selected from the group consisting of a circle, an oval, an ellipse, a square, a rectangle, a diamond, a parallelogram, a trapezoid, a rhombus, a convex kite, a concave kite, a pentagon, a hexagon, and an octagon, and a diameter or largest cross-sectional length that is in the range of 4-11 mm.

56. (canceled)

57. The microfluidic device of claim 50, wherein a ratio of the width at the wide first end of the tapering flow channel to the width at the narrow distal second end of tapering flow channel is from 2.75:1 to 3.25:1.

58. The microfluidic device of claim 50, wherein:
the detection point has a shape selected from the group consisting of a circle, an oval, an ellipse, a square, a rectangle, a diamond, a parallelogram, a trapezoid, a rhombus, a convex kite, a concave kite, a pentagon, a hexagon, and an octagon, and a diameter or largest cross-sectional length that is about 1.5 times to about 4.4 times the width of the widest flow path to which it is connected; and
the control point has a shape selected from the group consisting of a circle, an oval, an ellipse, a square, a rectangle, a diamond, a parallelogram, a trapezoid, a rhombus, a convex kite, a concave kite, a pentagon, a hexagon, and an octagon, and a diameter or largest cross-sectional length that is about 1.5 times to about 4.4 times the width of the widest flow path to which it is connected.

59.-68. (canceled)

69. A method of fabricating a paper-based microfluidic device, the method comprising:
providing a paper-based substrate having a first side and a reverse second side;
treating the first side of the paper-based substrate with a high energy plasma generated using radio frequency (RF) electromagnetic radiation at 8-12 MHz for a time period of about 3 to 6 minutes to produce a substrate having a plasma-treated first side;
printing, with a solid ink printer, a solid wax ink on the plasma-treated first side of the paper-based substrate to form a design of the microfluidic device on the plasma-treated first side of the paper-based substrate to yield a printed substrate;
heating the printed substrate to a temperature above the melting point of the wax contained in the solid wax ink to melt the wax;
allowing the melted wax to penetrate into at least a portion of the paper-based substrate; and
cooling the printed substrate to yield the microfluidic device.

70. The method of claim 69, wherein the heating is performed by:
inserting the printed substrate into a heated chamber set at a temperature in the range of 60° C.-150° C.; or
contacting the second side of the paper-based substrate with a heated surface set at a temperature in the range of 60° C.-150° C.

71. The method of claim 69, wherein the heating is performed for a time period of about 1 to 60 second(s).

72. The method of claim 69, wherein the design of the microfluidic device comprises:
a conjugate pad well;
a tapering flow channel having a wide first end and a narrow distal second end, the tapering flow channel attached via its wide first end to the conjugate pad well;
a detection point attached to the narrow distal second end of tapering flow channel;
a widening flow channel having a narrow first end and a wide distal second end, the narrow first end attached to the detection point;
a control point attached to the wide distal second end of the widening flow channel;
a discharge channel having a first end and a second end, and attached via the first end to the control point; and
an absorbent pad well attached to the second end of the discharge channel.

73. The method of claim 72, further comprising:
placing an absorbent pad into absorbent pad well; and
placing a conjugate pad in conjugate pad well.

74.-84. (canceled)

85. The method of claim 69, further comprising treating one or a combination of the tapering flow channel, the widening flow channel, and the discharge channel with a blocking agent.

86. The method of claim 85, wherein the blocking agent comprises bovine serum albumin.

87. A method of detecting cystatin C in a biological fluid sample to diagnose or monitor a chronic kidney disease, the method comprising:
providing a paper-based microfluidic device comprising:
a paper-based substrate; and
on a surface of the paper-based substrate:
a conjugate pad well;
a tapering flow channel having a wide first end and a narrow distal second end, the tapering flow channel attached via its wide first end to the conjugate pad well;
a detection point attached to the narrow distal second end of the tapering flow channel;
a widening flow channel having a narrow first end and a wide distal second end, the narrow first end attached to the detection point;
a control point attached to the wide distal second end of the widening flow channel;
a discharge channel having a first end and a second end, and attached via the first end to the control point; and
an absorbent pad well attached to the second end of the discharge channel;
treating the conjugate pad with a treatment solution;
loading a capture antibody at the detection point, wherein the capture antibody is an anti-cystatin C antibody;
loading a material that binds to a detection antibody at the control point;
blocking the tapering flow channel and the widening flow channel with a blocking solution and allowing the channels to dry at room temperature for a period of 1 to 24 hour(s);
rinsing the tapering flow channel and the widening flow channel with a rinsing solution and allowing the channels to dry at room temperature for a period of 1 to 30 minute(s);
placing the treated conjugate pad in the conjugate pad well;
loading the detection antibody and an aliquot of a biological fluid sample on the treated conjugate pad to produce a test fluid;
allowing the test fluid to traverse the length of the tapering flow channel, the widening flow channel, and the discharge channel;
acquiring an image of the detection point to calculate a first signal intensity, and acquiring an image of the control point to calculate a second signal intensity; and
using the first signal intensity and the second signal intensity to calculate a concentration of the cystatin C in the biological fluid sample.

88. The method of claim 87, wherein the biological fluid sample is blood or a blood fraction.

89.-93. (canceled)

94. The method of claim 87, wherein the detection antibody is a gold nanoparticle-labelled anti-cystatin C antibody.

95.-96. (canceled)

97. The method of claim 87, wherein the rinsing solution comprises a surfactant.

98. The method of claim 87, further comprising loading a washing solution onto the conjugate pad after allowing the test fluid to traverse the length of the tapering flow channel, the widening flow channel, and the discharge channel prior to acquiring an image.

99. The method of claim 98, further comprising allowing the tapering flow channel, the widening flow channel, and the discharge channel to dry after the washing solution has traversed their length prior to acquiring an image.

100. (canceled)

101. A kit, comprising:
the microfluidic device of claim 50;
instructions for the use thereof; and
a detection antibody.

102. (canceled)

103. (canceled)
